(19) United States
(12) Patent Application Publication
(10) Pub. No.: US 2025/0170128 A1
(43) Pub. Date: May 29, 2025

(54) Obicetrapib and SGLT2 Inhibitor Combination

(71) Applicant: NEWAMSTERDAM PHARMA B.V. (Dutch Chamber of Commerce No. 5597 1946), Naarden (NL)

(72) Inventors: Michael Harvey Davidson, Sunny Isles, FL (US); Sheng Cui, Lexington, MA (US)

(21) Appl. No.: 18/725,746

(22) PCT Filed: Dec. 28, 2022
(86) PCT No.: PCT/US2022/054160

§ 371 Date: Jun. 28, 2024

Related U.S. Application Data
(60) Provisional application No. 63/295,276, filed on Dec. 30, 2021.

(51) Int. Cl. A61K 31/506; A61K 9/20; A61K 9/28; A61K 31/351; A61K 31/357; A61K 31/381

(52) U.S. Cl. CPC A61K 31/506; A61K 9/2009; A61K 9/2013; A61K 9/2018; A61K 9/2054; A61K 9/2059; A61K 9/2813; A61K 9/282; A61K 9/2826; A61K 9/2866; A61K 31/351; A61K 31/357; A61K 31/381

(57) ABSTRACT

Provided herein is a pharmaceutical composition comprising obicetrapib, or a pharmaceutically acceptable salt thereof, and an SGTL2 inhibitor, or a pharmaceutically acceptable salt thereof. Also provided are dosage forms including the same. In some embodiments, the dosage form is a solid dosage form, such as a tablet. Also provided are processes for the preparation of fixed dose formulations of obicetrapib and an SGTL2 inhibitor, and methods for using the same in the treatment of a metabolic disorder (e.g., type 2 diabetes). Also provided are methods of treating or preventing a metabolic disorder in a subject who has or is at risk of developing a metabolic disorder, comprising: administering a therapeutically effective amount of obicetrapib, or a pharmaceutically acceptable salt thereof; and a therapeutically effective amount of at least one SGLT2 inhibitor, or a pharmaceutically acceptable salt thereof.

### 1. CROSS-REFERENCE TO RELATED APPLICATIONS

This application claims the benefit of and priority to U.S. provisional No. 63/295,276 filed Dec. 30, 2021, the disclosure of which is incorporated herein by reference in its entirety.

### 2. INTRODUCTION

Type 2 diabetes is an increasingly prevalent disease. Due to a high frequency of complications, type 2 diabetes leads to a significant reduction of life expectancy. Type 2 diabetes also causes significant morbidity. Because of diabetes-associated microvascular complications, type 2 diabetes is currently the most frequent cause of adult-onset loss of vision, renal failure, and amputations in the industrialized world. In addition, the presence of type 2 diabetes is associated with a two to five-fold increase in cardiovascular disease risk.

It is now widely accepted that glycemic control makes a difference in type II diabetes patients. The goal of diabetes therapy today is to achieve and maintain as near normal glycemia as possible to prevent the long-term microvascular and macrovascular complications associated with elevated glucose in the blood. Oral therapeutic options for the treatment of type II diabetes mellitus include compounds known as: sulfonylureas, biguanides (metformin), thiazolidinediones, and alpha-glucosidase inhibitors. The active agents from each class are generally administered to patients alone. However, once monotherapy becomes inadequate, combination therapy is feasible, despite the known side effect of weight gain associated with sulfonylurea and thiazolidinone therapies.

After long duration of disease, most patients with type 2 diabetes will eventually fail on oral therapy and become insulin-dependent, with the necessity for daily injections and multiple daily glucose measurements.

Thus, there is a need for alternative therapies that can better treat Type 2 diabetes and delay progression to insulin-dependence in type 2 diabetes and similar metabolic disorders.

### 3. SUMMARY OF THE INVENTION

The present disclosure provides a pharmaceutical composition comprising obicetrapib, or a pharmaceutically acceptable salt thereof, and an SGTL2 inhibitor, or a pharmaceutically acceptable salt thereof. Also provided are pharmaceutical dosage forms including the same. In some embodiments, the dosage form is a solid dosage form, such as a tablet. Also provided are processes for the preparation of fixed dose formulations of obicetrapib and an SGTL2 inhibitor, and methods for using the same in the treatment of a metabolic disorder (e.g., type 2 diabetes). Also provided are methods of treating or preventing a metabolic disorder in a subject who has or is at risk of developing a metabolic disorder, comprising: administering a pharmaceutical composition or pharmaceutical dosage form comprising obicetrapib, or a pharmaceutically acceptable salt thereof, and an SGTL2 inhibitor, or a pharmaceutically acceptable salt thereof. Also provided are methods of treating or preventing a metabolic disorder in a subject who has or is at risk of developing a metabolic disorder, comprising: administering a therapeutically effective amount of obicetrapib, or a pharmaceutically acceptable salt thereof; and a therapeutically effective amount of at least one SGLT2 inhibitor, or a pharmaceutically acceptable salt thereof.

A first aspect of this disclosure includes a pharmaceutical composition comprising:
a) a therapeutically effective amount of obicetrapib, or a pharmaceutically acceptable salt thereof; and
b) a therapeutically effective amount of at least one SGLT2 inhibitor, or a pharmaceutically acceptable salt thereof.

A second aspect of this disclosure includes a pharmaceutical dosage form including a pharmaceutical composition comprising obicetrapib and an SGLT2 inhibitor (e.g., as described herein).

A third aspect of this disclosure includes a method of treating or preventing a metabolic disorder, comprising administering to a subject having or at risk of developing a metabolic disorder a therapeutically effective amount of a pharmaceutical composition comprising obicetrapib and an SGLT2 inhibitor (e.g., as described herein).

A fourth aspect of this disclosure includes a method of treating or preventing a metabolic disorder in a subject who has or is at risk of developing a metabolic disorder, comprising: administering a therapeutically effective amount of obicetrapib, or a pharmaceutically acceptable salt thereof; and a therapeutically effective amount of at least one SGLT2 inhibitor, or a pharmaceutically acceptable salt thereof.

In certain embodiments, the metabolic disorder is type 1 diabetes mellitus, type 2 diabetes mellitus, impaired glucose tolerance (IGT), impaired fasting blood glucose (IFG), hyperglycemia, postprandial hyperglycemia, obesity, or metabolic syndrome.

### 4. DETAILED DESCRIPTION OF THE INVENTION

As summarized above, in one aspect, this disclosure provides a pharmaceutical composition comprising obicetrapib, or a pharmaceutically acceptable salt thereof, and an SGTL2 inhibitor, or a pharmaceutically acceptable salt thereof. Also provided are pharmaceutical dosage forms including the same. In some embodiments, the dosage form is a solid dosage form, such as a tablet. Also provided are processes for the preparation of fixed dose combination formulations of obicetrapib and an SGTL2 inhibitor, and methods for using the same in the treatment of a metabolic disorder (e.g., type 2 diabetes). Also provided are methods of treating or preventing a metabolic disorder in a subject who has or is at risk of developing a metabolic disorder, comprising: administering a therapeutically effective amount of obicetrapib, or a pharmaceutically acceptable salt thereof; and a therapeutically effective amount of at least one SGLT2 inhibitor, or a pharmaceutically acceptable salt thereof.

The pharmaceutical compositions of this disclosure are described in greater detail below. The obicetrapib active compound is described, and SGLT2 inhibitors are described. Also described are pharmaceutical compositions including obicetrapib and a SGTL2 inhibitor, pharmaceutical dosage forms comprising obicetrapib and SGLT2 inhibitors, and processes for the preparation of fixed dose formulations of the same. Methods in which the pharmaceutical compositions of this disclosure find use are also described.

### 4.1. Pharmaceutical Compositions

As summarized above, this disclosure provides a pharmaceutical composition comprising obicetrapib, or a pharmaceutically acceptable salt thereof, and an SGTL2 inhibitor, or a pharmaceutically acceptable salt thereof.

### 4.1.1. Obicetrapib Compounds

The pharmaceutical compositions disclosed herein include obicetrapib, or a pharmaceutically acceptable salt thereof. More specifically, obicetrapib, or (2R,4S)-4-{[3,5-bis(trifluoromethyl)benzyl]-[5-(3-carboxypropoxy)pyrimidin-2-yl]amino}-2-ethyl-6-trifluoromethyl-3,4-dihydro-2H-quinoline-1-carboxylic acid ethyl ester, is a cholesteryl ester transfer protein (CETP) inhibitor of formula (I), wherein Et represents an ethyl group:

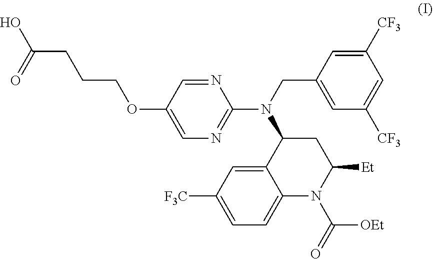 [SMILES: CCOC(=O)N1c2ccc(C(F)(F)F)cc2[C@@H](N(Cc2cc(C(F)(F)F)cc(C(F)(F)F)c2)c2ncc(OCCCC(=O)O)cn2)C[C@H]1CC]

Compared to other known CETP-inhibitors, only a relatively low dose of the compound of Formula (I) is needed to reach near complete CETP inhibition. Typically, repeated daily dosages (once a day) as low as 2.5 mg of the compound of Formula (I) have been shown to be sufficient to reach near complete CETP-inhibition. These are considerably lower dosages than had to be used for other CETP-inhibitors. Moreover, clinical studies have also shown that the compound of Formula (I) is well tolerated and that it does not lead to serious side effects.

Inhibiting CETP can reduce LDL-C and elevates high-density lipoprotein cholesterol (HDLC) levels. CETP is a plasma protein secreted primarily by liver and adipose tissue. CETP mediates the transfer of cholesteryl esters from HDL to apolipoprotein B (Apo B)-containing particles (mainly LDL and very low density lipoprotein VLDL) in exchange for triglycerides, thereby decreasing the cholesterol content in HDL in favor of that in (V) LDL. Hence, CETP inhibition has been hypothesized to retain cholesteryl esters in HDL-C and decrease the cholesterol content of the atherogenic Apo B fraction.

In certain embodiments, the obicetrapib is in the form of a pharmaceutically acceptable salt. As used herein, “a pharmaceutically acceptable salt” includes any salt that retains the activity of the active agent(s) and is acceptable for pharmaceutical use. A pharmaceutically acceptable salt also refers to any salt which may form in vivo as a result of administration of an acid, another salt, or a prodrug which is converted into an acid or salt. The pharmaceutically acceptable salt of the disclosed compounds may be prepared by methods well known to those skilled in the art.

The pharmaceutically acceptable salts of the compound (I) may include, for example, alkali metal salts such as lithium, sodium or potassium salt; alkali earth metal salts such as calcium or magnesium salt; salts with zinc or aluminum; salts with organic bases such as ammonium, choline, diethanolamine, lysine, ethylenediamine, tert-butylamine, tert-octylamine, tris(hydroxymethyl)aminomethane, N-methylglucosamie, triethanolamine or dehydroabiethylamine; salts with inorganic acids such as hydrochloric acid, hydrobromic acid, hydroiodic acid, sulfuric acid, nitric acid or phosphoric acid; salts with organic acids such as formic acid, acetic acid, propionic acid, oxalic acid, malonic acid, succinic acid, fumaric acid, maleic acid, lactic acid, malic acid, tartaric acid, citric acid, methanesulfonic acid, ethanesulfonic acid, benzenesulfonic acid or toluenesulfonic acid; or salts derived from acidic amino acids such as aspartic acid or glutamic acid.

Additionally, the pharmaceutically acceptable salts of the compound (I) may include, for example, quaternary salts formed between a compound of the formula (I) and an alkyl halide or phenylalkyl halide.

Furthermore, the compositions can comprise obicetrapib in the form of a solvate, comprising a pharmaceutically acceptable solvent, such as water (‘hydrate’), ethanol, and the like. In general, the solvated forms are considered equivalent to the unsolvated forms for the purposes of the present disclosure.

In certain embodiments, the obicetrapib is a calcium salt.

Obicetrapib has previously been described (see, for example, WO 2005/095409A2). It can be prepared by methods described therein (e.g., see WO 2005/095409A2 and U.S. Pat. Nos. 7,872,126 and 8,158,640, Examples 1 and 177-180), or by the methods described in WO 2007/116922 A1 and U.S. Pat. No. 8,084,611, or by the methods described in WO 2016/024858 and U.S. Pat. No. 10,112,904, the disclosures of each of which is incorporated herein by reference in its entirety.

Obicetrapib is present in the pharmaceutical composition in a therapeutically effective amount. With respect to obicetrapib, a “therapeutically effective amount” refers to an amount effective to lower low density lipoprotein-cholesterol (LDL-C). Without intending to be bound by theory, it is believed that lower LDL-C reduces lipid toxicity on pancreatic beta cells. In some embodiments, a “therapeutically effective amount” of obicetrapib is an amount that, when administered to an individual in one or more doses, in combination therapy (e.g., as described herein with an SGLT2 inhibitor), is effective to lower LDL-C in the subject by about 5%, at least about 10%, at least about 20%, at least about 30%, at least about 40%, at least about 50%, at least about 80%, at least about 90%, or at least about 95%, compared to LDL-C level in the individual in the absence of, or before, treatment with the combination.

In some embodiments, the pharmaceutical composition comprises from about 1% to about 25% w/w of obicetrapib, or a salt, solvate or hydrate thereof. In further embodiments, the composition comprises from about 1% to about 20% w/w, or from about 1% to about 15% w/w, or from about 1% to about 10% w/w, or from about 5% to about 15% w/w, or from about 5% to about 12% w/w of obicetrapib, or a salt, solvate or hydrate thereof. In further embodiments, the pharmaceutical composition comprises about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, or about 15% w/w of obicetrapib, or a salt, solvate or hydrate thereof. In a specific embodiment, the pharmaceutical composition comprises about 5% w/w of obicetrapib, or a salt, solvate or hydrate thereof. In a specific embodiment, the pharmaceutical composition comprises about 10% w/w of obicetrapib, or a salt, solvate or hydrate thereof.

In some embodiments, the pharmaceutical composition comprises from 1% to 25% w/w of obicetrapib, or a salt, solvate or hydrate thereof. In further embodiments, the composition comprises from 1% to 20% w/w, or from 1% to 15% w/w, or from 1% to 10% w/w, or from 5% to 15% w/w, or from 5% to 12% w/w of obicetrapib, or a salt, solvate or hydrate thereof. In further embodiments, the pharmaceutical composition comprises 1% w/w, 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w, or 15% w/w of obicetrapib, or a salt, solvate or hydrate thereof. In a specific embodiment, the pharmaceutical composition comprises 5% w/w of obicetrapib, or a salt, solvate or hydrate thereof. In a specific embodiment, the pharmaceutical composition comprises 10% w/w of obicetrapib, or a salt, solvate or hydrate thereof.

### 4.1.2. SGLT2 Inhibitors

The pharmaceutical compositions disclosed herein also include a SGTL2 inhibitor, or a pharmaceutically acceptable salt thereof.

The term “SGLT2 inhibitor” refers to a compound, in particular to a glucopyranosyl-derivative, i.e. compound having a glucopyranosyl-moiety, which shows an inhibitory effect on the sodium-glucose transporter 2 (SGLT2), in particular the human SGLT2. In some embodiments, the activity of the SGLT2 inhibitor is determined by an inhibition assay, e.g., by an assay that determines the level of activity of the enzyme either in a cell-free system or in a cell after treatment with a subject compound, relative to a control, by measuring the IC₅₀ or EC₅₀ value, respectively. In certain embodiments, the SGLT2 inhibitor has an IC₅₀ value (or EC₅₀ value) of 10 μM or less, such as 3 μM or less, 1 μM or less, 500 nM or less, 300 nM or less, 200 nM or less, 100 nM or less, 50 nM or less, 30 nM or less, 10 nM or less, 5 nM or less, 3 nM or less, 1 nM or less, or even lower. The inhibitory effect on human SGLT2 can be determined by methods known in the literature, in particular as described in the application WO 2005/092877 or WO 2007/093610 (pages 23/24), which are incorporated herein by reference in their entirety. The term “SGLT2 inhibitor” also comprises any pharmaceutically acceptable salts thereof, hydrates and solvates thereof, including the respective crystalline forms.

In certain embodiments, the SGLT2 inhibitor is selected from canagliflozin, dapagliflozin, ertugliflozin, empagliflozin, bexagliflozin, tofogliflozin, ipragliflozin, luseogliflozin, remogliflozin, remogliflozin etabonate, sergliflozin, sergliflozin etabonate, atigliflozin, and sotagliflozin.

In certain embodiments, the SGLT2 inhibitor is selected from canagliflozin, dapagliflozin, ertugliflozin, empagliflozin, bexagliflozin, tofogliflozin, ipragliflozin, luseogliflozin, remogliflozin etabonate, sergliflozin etabonate, and sotagliflozin.

In certain embodiments, the SGLT2 inhibitor is selected from canagliflozin, dapagliflozin, ertugliflozin, and empagliflozin.

In some embodiments, the SGLT2 inhibitor is empagliflozin.

In some embodiments, the SGLT2 inhibitor is dapagliflozin.

In some embodiments, the SGLT2 inhibitor is canagliflozin.

In some embodiments, the SGLT2 inhibitor is ertugliflozin.

In certain embodiments, the SGLT2 inhibitor is a glucopyranosyl-substituted benzene derivatives of the formula (II):

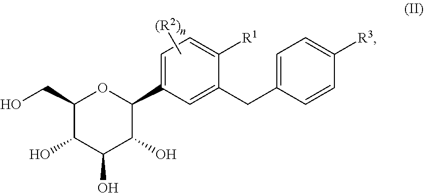

or a hydrate, solvent, or pharmaceutically acceptable salt thereof,
wherein:
R¹ is halogen (e.g., chloro), (C₁₋₃)alkyl, or cyano;
each R² is independently H, (C₁₋₃)alkyl, (C₁₋₃)alkoxy or hydroxy;
R³ is (C₁₋₃)alkyl, cycloalkyl, alkynyl, (C₁₋₃)alkoxy, (R)-tetrahydrofuran-3-yloxy or (S)-tetrahydrofuran-3-yloxy; and
n is 0 to 3.

In certain embodiments, the SGLT2 inhibitor is a prodrug of any of the beforementioned SGLT2 inhibitors.

In certain embodiments, the SGLT2 inhibitor of formula (II) and methods of their synthesis are described, for example, in the following international patent applications: WO 2005/092877, WO 2006/117360, WO 2006/117359, WO 2006/120208, WO 2006/064033, WO 2007/031548, WO 2007/093610, WO 2008/020011, WO 2008/055870, the disclosures of which are incorporated herein by reference.

In some embodiments of formula (II), R¹ is methyl. In some cases, R¹ is halogen. In certain cases, the halogen is chloride. In some other cases, R¹ is cyano.

In some embodiments of formula (II), each R² is H. In certain some embodiments, n is 1 and R² is methyl. In certain embodiments, n is 1 and R² is methoxy. In certain embodiments, n is 1 and R² is hydroxy.

In certain embodiments of formula (II), R³ is ethyl, cyclopropyl, ethynyl, (R)-tetrahydrofuran-3-yloxy or (S)-tetrahydrofuran-3-yloxy. In some cases, R³ is cyclopropyl, ethynyl, (R)-tetrahydrofuran-3-yloxy or (S)-tetrahydrofuran-3-yloxy. In certain cases, R³ is ethynyl, (R)-tetrahydrofuran-3-yloxy or (S)-tetrahydrofuran-3-yloxy.

In certain cases, the SGLT2 inhibitor is selected from any one of the following compounds:

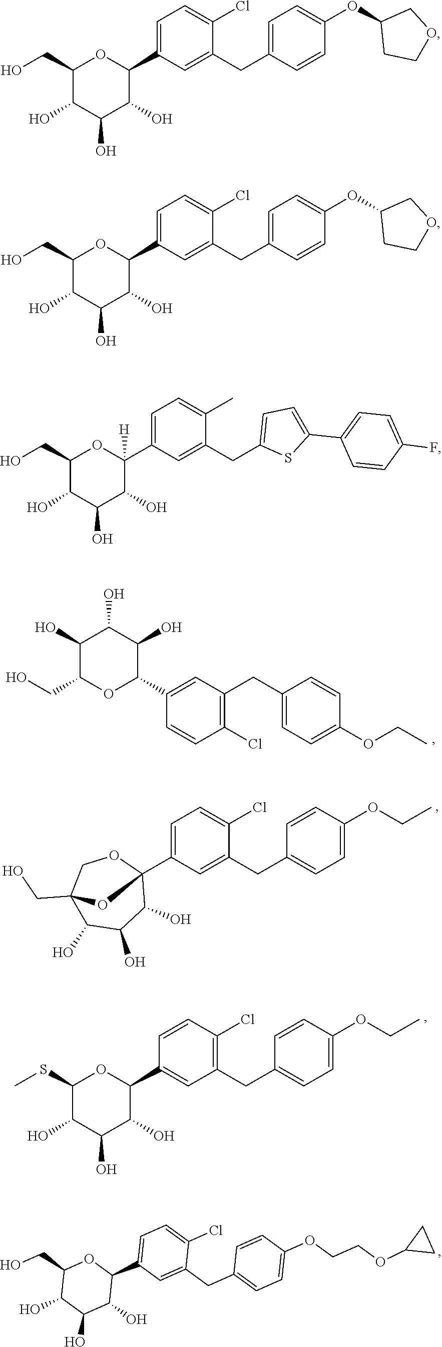

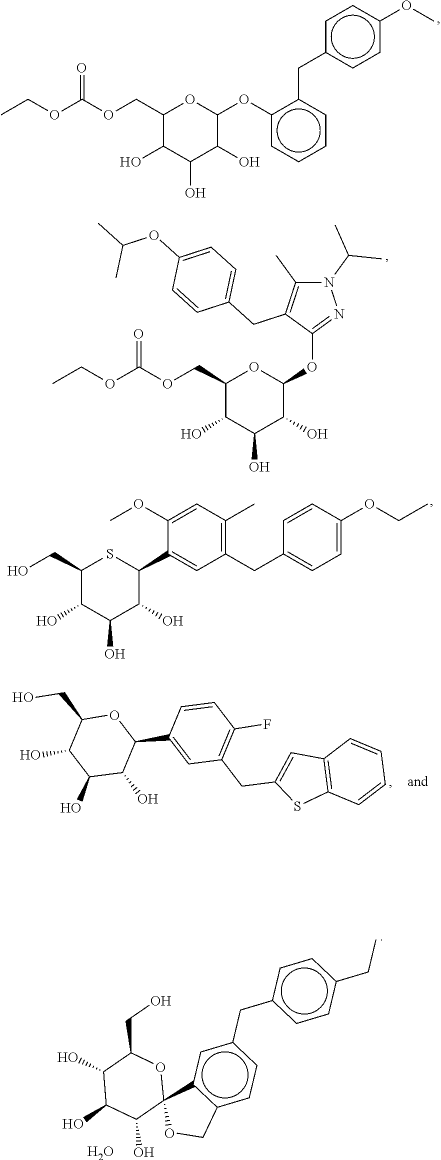

It is to be understood that the definitions of the above listed SGLT2 inhibitors, including the glucopyranosyl-substituted benzene derivatives of the formula (II), also comprise their hydrates, solvates, polymorphic forms, and prodrugs thereof. The term “empagliflozin” as used herein refers to empagliflozin, including hydrates, solvates, and crystalline forms thereof. In one embodiment, the SGLT2 inhibitor is in the crystalline form as described in the international patent application WO 2006/117360, which hereby is incorporated herein in its entirety. In certain some embodiments, the SGLT2 inhibitor is in the crystalline form as described in the international patent application WO 2006/117359, which hereby is incorporated herein in its entirety. In certain some embodiments, the SGLT2 inhibitor is in the crystalline form as described in the international patent application WO 2008/049923, which hereby is incorporated herein in its entirety. These crystalline forms possess good solubility properties which can enable good bioavailability of the SGLT2 inhibitor. Furthermore, the crystalline forms are physico-chemically stable and thus provide a good shelf-life stability of the pharmaceutical composition.

The term “dapagliflozin” as used herein refers to dapagliflozin, including hydrates, solvates, and crystalline forms thereof. The compound and methods of its synthesis are described in WO 03/099836 for example. Hydrates, solvates and crystalline forms are described in the patent applications WO 2008/116179 and WO 2008/002824, for example.

The term “canagliflozin” as used herein refers to canagliflozin, including hydrates, solvates, and crystalline forms thereof. The compound and methods of its synthesis are described in WO 2005/012326 and WO 2009/035969, for example. Certain hydrates, solvates and crystalline forms are described in the patent applications WO 2008/069327, for example.

The term “atigliflozin” as used herein refers to atigliflozin, including hydrates, solvates, and crystalline forms thereof. The compound and methods of its synthesis are described in WO 2004/007517, for example.

The term “ipragliflozin” as used herein refers to ipragliflozin, including hydrates, solvates, and crystalline forms thereof. The compound and methods of its synthesis are described in WO 2004/080990, WO 2005/012326 and WO 2007/114475, for example.

The term “tofogliflozin” as employed herein refers to tofogliflozin, including hydrates, solvates, and crystalline forms thereof. The compound and methods of its synthesis are described in WO 2007/140191 and WO 2008/013280, for example.

The term “remogliflozin” as used herein refers to remogliflozin and prodrugs of remogliflozin, in particular remogliflozin etabonate, including hydrates, solvates, and crystalline forms thereof. Methods of its synthesis are described in the patent applications EP 1213296 and EP 1354888, for example.

The term “sergliflozin” as employed herein refers to sergliflozin and prodrugs of sergliflozin, in particular sergliflozin etabonate, including hydrates, solvates, and crystalline forms thereof. Methods for its manufacture are described in the patent applications EP 1344780 and EP 1489089, for example.

The disclosure of each of the foregoing documents cited above in connection with the specified SGLT2 inhibitors is specifically incorporated herein by reference in its entirety.

In some embodiments, the pharmaceutical composition comprises a therapeutically effective amount of a SGLT2 inhibitor (e.g., as described herein). With respect to the SGLT2 inhibitor, a “therapeutically effective amount” refers to an amount effective to inhibit SGLT2, and/or lower glucose reabsorption. Without intending to be bound by theory, it is believed that lowering glucose absorption reduces blood glucose levels, which lower glucose toxicity of pancreatic beta cells. In some embodiments, a “therapeutically effective amount” of an SGLT2 inhibitor is an amount that, when administered to an individual in one or more doses, in combination therapy (e.g., as described herein with obicetrapib), is effective to lower glucose reabsorption in the subject by about 5%, at least about 10%, at least about 20%, at least about 30%, at least about 40%, at least about 50%, at least about 80%, at least about 90%, or at least about 95%, compared to glucose reabsorption level in the individual in the absence of treatment with the combination, or alternatively, compared to the glucose level in the subject before or after treatment with the combination.

In some embodiments, the pharmaceutical composition comprises from about 1% to about 75% w/w of the SGLT2 inhibitor, or a salt, solvate or hydrate thereof. In further embodiments, the pharmaceutical composition comprises from about 1% to about 50% w/w, or from about 1% to about 40% w/w, about 1% to about 30% w/w, about 1% to about 20%, about 1% to 10%, or from about 5% to about 15% w/w, or from about 10% to about 25% w/w of the SGLT2 inhibitor, or a salt, solvate or hydrate thereof. In further embodiments, the pharmaceutical composition comprises about 5% w/w, about 10% w/w, about 15% w/w, about 20% w/w, about 25% w/w, about 30% w/w, about 35% w/w, about 40% w/w, about 45% w/w, about 50% w/w, about 55% w/w, about 60% w/w, about 65% w/w, about 70% w/w, about 75% w/w, of the SGLT2 inhibitor, or a salt, solvate or hydrate thereof. In a specific embodiment, the pharmaceutical composition comprises about 5% w/w of the SGLT2 inhibitor, or a salt, solvate or hydrate thereof. In a specific embodiment, the pharmaceutical composition comprises about 10% w/w of the SGLT2 inhibitor, or a salt, solvate or hydrate thereof. In a specific embodiment, the pharmaceutical composition comprises about 15% w/w of the SGLT2 inhibitor, or a salt, solvate or hydrate thereof. In a specific embodiment, the pharmaceutical composition comprises about 25% w/w of the SGLT2 inhibitor, or a salt, solvate or hydrate thereof. In a specific embodiment, the pharmaceutical composition comprises about 50% w/w of the SGLT2 inhibitor, or a salt, solvate or hydrate thereof. In a specific embodiment, the pharmaceutical composition comprises about 75% w/w of the SGLT2 inhibitor, or a salt, solvate or hydrate thereof.

In some embodiments, the pharmaceutical composition comprises from 1% to 75% w/w of the SGLT2 inhibitor, or a salt, solvate or hydrate thereof. In further embodiments, the pharmaceutical composition comprises from 1% to 50% w/w, or from 1% to 40% w/w, 1% to 30% w/w, 1% to 20%, 1% to 10%, or from 5% to 15% w/w, or from 10% to 25% w/w of the SGLT2 inhibitor, or a salt, solvate or hydrate thereof. In further embodiments, the pharmaceutical composition comprises 5% w/w, 10% w/w, 15% w/w, 20% w/w, 25% w/w, 30% w/w, 35% w/w, 40% w/w, 45% w/w, 50% w/w, 55% w/w, 60% w/w, 65% w/w, 70% w/w, 75% w/w, of the SGLT2 inhibitor, or a salt, solvate or hydrate thereof. In a specific embodiment, the pharmaceutical composition comprises 5% w/w of the SGLT2 inhibitor, or a salt, solvate or hydrate thereof. In a specific embodiment, the pharmaceutical composition comprises 10% w/w of the SGLT2 inhibitor, or a salt, solvate or hydrate thereof. In a specific embodiment, the pharmaceutical composition comprises 15% w/w of the SGLT2 inhibitor, or a salt, solvate or hydrate thereof. In a specific embodiment, the pharmaceutical composition comprises 25% w/w of the SGLT2 inhibitor, or a salt, solvate or hydrate thereof. In a specific embodiment, the pharmaceutical composition comprises 50% w/w of the SGLT2 inhibitor, or a salt, solvate or hydrate thereof. In a specific embodiment, the pharmaceutical composition comprises 75% w/w of the SGLT2 inhibitor, or a salt, solvate or hydrate thereof.

### 4.1.3. Additional Active Agents

The pharmaceutical compositions disclosed herein may optionally include one or more additional active agents. In some embodiments, at least one of the one or more additional active agents is an antidiabetic agent. In certain embodiments, at least of the one or more additional actives is an antidiabetic agent selected from biguanides, thiazolidindiones, sulfonylureas, glinides, inhibitors of alpha-glucosidase, insulin, DPP-4 inhibitors and amylin analogs, including pharmaceutically acceptable salts of the beforementioned agents.

In some embodiments, the combination of obicetrapib, a SGLT2 inhibitor, and one or more additional active agents according to the present disclosure can allow for a reduction in the dose of either the obicetrapib or the SGLT2.

A dose reduction can be beneficial for patients which otherwise would potentially suffer from side effects in a therapy using a higher dose of one or more of the active ingredients. As such, the pharmaceutical composition and methods according to the present disclosure can exhibit less side effects to a corresponding monotherapy with any of the active agents (e.g., obicetrapib or the SGLT2 inhibitor), thereby making the therapy more tolerable and improving an individual's compliance with the treatment.

In certain embodiments, the additional active agent is a biguanides. Examples of biguanides include metformin, phenformin and buformin. In certain cases, the additional active agent is metformin. The term “metformin” as employed herein refers to metformin or a pharmaceutically acceptable salt thereof such as the hydrochloride salt, the metformin (2:1) fumarate salt, and the metformin (2:1) succinate salt, the hydrobromide salt, the p-chlorophenoxy acetate or the embonate, and other known metformin salts of mono and dibasic carboxylic acids. It one embodiment, the metformin employed herein is the metformin hydrochloride salt.

In certain embodiments, the pharmaceutical composition comprises from 1 to 50% w/w of metformin hydrochloride salt, such as 1 to 45% w/w, 1 to 40% w/w, 1 to 35% w/w, 1 to 30% w/w, 1 to 25% w/w, 1 to 20% w/w, 1 to 15% w/w, 1 to 10% w/w, or 1 to 5% w/w of metformin hydrochloride salt.

In certain embodiments, the additional active agent is a DPP-4 inhibitor. Examples of DPP-4 inhibitors are linagliptin, sitagliptin, vildagliptin, saxagliptin, denagliptin, alogliptin, carmegliptin, melogliptin, dutogliptin, including pharmaceutically acceptable salts, hydrates and solvates thereof. In certain embodiments, the DPP-4 inhibitor is linagliptin.

In certain embodiments, the pharmaceutical composition comprises from 1 to 10% w/w of linagliptin, such as 1 to 9% w/w, 1 to 8% w/w, 1 to 7% w/w, 1 to 6% w/w, 1 to 5% w/w, 2 to 5% w/w, 2 to 4% w/w, of linagliptin, or a salt, solvate or hydrate thereof. In certain cases, the pharmaceutical composition comprises 2.5% w/w of linagliptin, or a salt, solvate or hydrate thereof. In certain cases, the pharmaceutical composition comprises 5% w/w of linagliptin, or a salt, solvate or hydrate thereof.

In certain embodiments, the additional active agent is a thiazolidindione. Examples of thiazolidindiones (TZD) includes pioglitazone and rosiglitazone. The term “pioglitazone” as employed herein refers to pioglitazone, including its enantiomers, mixtures thereof and its racemate, or a pharmaceutically acceptable salt thereof such as the hydrochloride salt. The term “rosiglitazone” as employed herein refers to rosiglitazone, including its enantiomers, mixtures thereof and its racemate, or a pharmaceutically acceptable salt thereof such as the maleate salt.

In certain embodiments, the additional active agent is a sulfonylurea. Examples of sulfonylureas are glibenclamide, tolbutamide, glimepiride, glipizide, gliquidone, glibornuride, glyburide, glisoxepide and gliclazide. In certain cases, the sulfonylurea is selected from tolbutamide, gliquidone, glibenclamide, glipizide and glimepiride. In certain cases, the sulfonylurea is selected from glibenclamide, glipizide and glimepiride. Each term of the group “glibenclamide”, “glimepiride”, “gliquidone”, “glibornuride”, “gliclazide”, “glisoxepide”, “tolbutamide” and “glipizide” as employed herein refers to the respective active drug or a pharmaceutically acceptable salt thereof.

In certain embodiments, the additional active agent is a glinide. Examples of glinides are nateglinide, repaglinide and mitiglinide. The term “nateglinide” as employed herein refers to nateglinide, including its enantiomers, mixtures thereof and its racemate, or pharmaceutically acceptable salts and esters thereof. The term “repaglinide” as employed herein refers to repaglinide, including its enantiomers, mixtures thereof and its racemate, or pharmaceutically acceptable salts and esters thereof.

In certain embodiments, the additional active agent is an inhibitor of alpha-glucosidase. Examples of inhibitors of alpha-glucosidase are acarbose, voglibose and miglitol. Each term of the group “acarbose”, “voglibose” and “miglitol” as employed herein refers to the respective active drug or a pharmaceutically acceptable salt thereof.

In certain embodiments, the additional active is an amylin analog. An example of an amylin analog is pramlintide, including pharmaceutically acceptable salts, hydrates and solvates thereof. For example pramlintide acetate is marketed under the tradename Symlin.

As such, according to a further embodiment, the pharmaceutical compositions disclosed herein include a combination of obicetrapib, an SGLT2 inhibitor and one or more further antidiabetic agents. In certain embodiments, the further antidiabetic agent is metformin, linagliptin, or a combination thereof.

### 4.1.4. Excipients

The pharmaceutical compositions provided in accordance with the present disclosure can be administered orally. In certain embodiments, the disclosure therefore provides pharmaceutical compositions that comprise obicetrapib and an SGLT2 inhibitor as described herein, and one or more pharmaceutically acceptable excipients or carriers including but not limited to, inert solid diluents and fillers, diluents, including sterile aqueous solution and various organic solvents, permeation enhancers, solubilizers, surfactants, disintegrants, lubricants, binders, glidants, adjuvants, and combinations thereof. Such compositions are prepared in a manner well known in the pharmaceutical art (see, e.g., Remington: The Science and Practice of Pharmacy (Remington: The Science and Practice of Pharmacy, 23rd Edition, ISBN-13:978-0128200070); and Modern Pharmaceutics, Marcel Dekker, Inc., 4th Ed. (G. S. Banker & C. T. Rhodes, Eds.).

The pharmaceutical compositions may be administered in a fixed dose combination by oral administration. Administration may be via capsule, tablet, or the like. In one embodiment, the obicetrapib and the SGLT2 inhibitor combination is in the form of a tablet. In a further embodiment, the tablet is a compressed tablet. In making the pharmaceutical compositions that include the solid described herein, the active ingredient is usually diluted by an excipient and/or enclosed within such a carrier that can be in the form of a capsule, tablet, sachet, or other container. When the excipient serves as a diluent, it can be in the form of a solid, semi-solid or liquid material (as above), which acts as a vehicle, carrier or medium for the active ingredient.

The pharmaceutical composition may be formulated for immediate release or sustained release. A “sustained release formulation” is a formulation which is designed to slowly release a therapeutic agent in the body over an extended period of time, whereas an “immediate release formulation” is a formulation which is designed to quickly release a therapeutic agent in the body over a shortened period of time. In some cases the immediate release formulation may be coated such that the therapeutic agent is only released once it reached the desired target in the body (e.g. the stomach). In a specific embodiment, the pharmaceutical composition is formulated for immediate release.

The pharmaceutical composition may further comprise pharmaceutical excipients such as diluents, binders, fillers, glidants, disintegrants, lubricants, solubilizers, and combinations thereof. Some examples of suitable excipients are described herein. When the pharmaceutical composition is formulated into a tablet, the tablet may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

In some embodiments, the pharmaceutical composition comprises a diluent selected from the group consisting of dicalcium phosphate, cellulose, microcrystalline cellulose, compressible sugars, dibasic calcium phosphate dehydrate, lactose, lactose monohydrate, mannitol, tribasic calcium phosphate, and combinations thereof. In certain cases, the diluent comprises microcrystalline cellulose. In certain cases, the diluent comprises mannitol. In certain cases, the diluent comprises lactose anhydrous or lactose monohydrate.

In some embodiments, the pharmaceutical composition comprises a controlled release matrix. In certain cases, the diluent is polyethylene oxide and hypromellose.

In further embodiments, the pharmaceutical composition comprises microcrystalline cellulose in an amount from about 1 to about 100% w/w, or from about 1 to about 80% w/w, or from about 1% to about 75% w/w, or from about 5 to about 75% w/w, or from about 10 to about 70% w/w, or from about 15 to about 70% w/w. In specific embodiments, the microcrystalline cellulose is present in an amount of about 5%, or about 10%, or about 15%, or about 20%, or about 25%, or about 30%, or about 35%, or about 40%, or about 45%, or about 50%, or about 55%, or about 60%, or about 65%, or about 70%, or about 75% w/w. In a further specific embodiment, the microcrystalline cellulose is in an amount of about 60% w/w. In a further specific embodiment, the microcrystalline cellulose is in an amount of about 65% w/w.

In further embodiments, the pharmaceutical composition comprises microcrystalline cellulose in an amount from 1 to 100% w/w, or from 1 to 80% w/w, or from 1% to 75% w/w, or from 5 to 75% w/w, or from 10 to 70% w/w, or from 15 to 70% w/w. In specific embodiments, the microcrystalline cellulose is present in an amount of 5%, or 10%, or 15%, or 20%, or 25%, or 30%, or 35%, or 40%, or 45%, or 50%, or 55%, or 60%, or 65%, or 70%, or 75% w/w. In a further specific embodiment, the microcrystalline cellulose is in an amount of 60% w/w. In a further specific embodiment, the microcrystalline cellulose is in an amount of 65% w/w.

In yet further embodiments, the pharmaceutical composition comprises mannitol in an amount from about 1 to about 40% w/w, or from about 1 to about 35% w/w, or from about 1% to about 25% w/w, or from about 5 to about 35% w/w, or from about 10 to about 30% w/w, or from about 15 to about 25% w/w. In specific embodiments, the mannitol is present in an amount of about 5%, or about 20%, or about 15%, or about 30%, or about 22%, or about 23%, or about 24%, or about 25% w/w. In a further specific embodiment, the microcrystalline cellulose is in an amount of about 20% w/w.

In yet further embodiments, the pharmaceutical composition comprises mannitol in an amount from 1 to 40% w/w, or from 1 to 35% w/w, or from 1% to 25% w/w, or from 5 to 35% w/w, or from 10 to 30% w/w, or from 15 to 25% w/w. In specific embodiments, the mannitol is present in an amount of 5%, or 20%, or 15%, or 30%, or 22%, or 23%, or 24%, or 25% w/w. In a further specific embodiment, the microcrystalline cellulose is in an amount of 20% w/w.

In some embodiments, the pharmaceutical composition comprises a disintegrant selected from the group consisting of croscarmellose sodium, crospovidone, modified corn starch, pregelatinized starch, sodium starch glycolate, and combinations thereof.

In certain embodiments, the pharmaceutical composition comprises sodium starch glycolate in an amount from about 1 to about 20% w/w, or from about 1 to about 15% w/w, or from about 1 to about 10% w/w, or from about 1 to about 8% w/w, or from about 2 to about 8% w/w. In specific embodiments, the croscarmellose sodium is present in an amount of about 1%, or about 3%, or about 4%, or about 5%, or about 6%, or about 7%, or about 10% w/w. In a further specific embodiment, the croscarmellose sodium is in an amount of about 5% w/w.

In certain embodiments, the pharmaceutical composition comprises sodium starch glycolate in an amount from 1 to 20% w/w, or from 1 to 15% w/w, or from 1 to 10% w/w, or from 1 to 8% w/w, or from 2 to 8% w/w. In specific embodiments, the croscarmellose sodium is present in an amount of 1%, or 3%, or 4%, or 5%, or 6%, or 7%, or 10% w/w. In a further specific embodiment, the croscarmellose sodium is in an amount of 5% w/w.

In some embodiments, the pharmaceutical composition comprises a glidant selected from the group consisting of colloidal silicon dioxide, talc, and combinations thereof.

In further embodiments, the pharmaceutical composition comprises colloidal silicon dioxide in an amount from about 0.1 to about 5% w/w, or from about 0.1 to about 4.5% w/w, or from about 0.1 to about 4% w/w, or from about 0.5 to about 5.0% w/w, or from about 0.5 to about 3% w/w, or from about 0.5 to about 2% w/w, or from about 0.5 to about 1.5% w/w. In specific embodiments, the colloidal silicon dioxide is present in an amount of about 0.1% w/w, 0.5% w/w, 0.75% w/w, 0.95% w/w, 1.0% w/w, or 1.2% w/w. In a further specific embodiment, the colloidal silicon dioxide is present in an amount of about 1% w/w.

In further embodiments, the pharmaceutical composition comprises colloidal silicon dioxide in an amount from 0.1 to 5% w/w, or from 0.1 to 4.5% w/w, or from 0.1 to 4% w/w, or from 0.5 to 5.0% w/w, or from 0.5 to 3% w/w, or from 0.5 to 2% w/w, or from 0.5 to 1.5% w/w. In specific embodiments, the colloidal silicon dioxide is present in an amount of 0.1% w/w, 0.5% w/w, 0.75% w/w, 0.95% w/w, 1.0% w/w, or 1.2% w/w. In a further specific embodiment, the colloidal silicon dioxide is present in an amount of 1% w/w.

In some embodiments, the pharmaceutical composition comprises a lubricant selected from the group consisting of calcium stearate, magnesium stearate, polyethylene glycol, sodium stearyl fumarate, stearic acid, and combinations thereof.

In further embodiments, the pharmaceutical composition comprises magnesium stearate in an amount from about 0.1 to about 3% w/w, or from about 0.1 to about 2.5% w/w, or from about 0.5 to about 3% w/w, or from about 0.5 to about 2.5% w/w, or from about 0.5 to about 2% w/w, or from about 1 to about 3% w/w, or from about 1 to about 2% w/w. In specific embodiments, the magnesium stearate is present in an amount of about 0.1%, or about 0.5, or about 0.7%, or about 0.9%, or about 1.0%, or about 1.2% w/w. In a further specific embodiment, the magnesium stearate is in an amount of about 1% w/w.

In further embodiments, the pharmaceutical composition comprises magnesium stearate in an amount from 0.1 to 3% w/w, or from 0.1 to 2.5% w/w, or from 0.5 to 3% w/w, or from 0.5 to 2.5% w/w, or from 0.5 to 2% w/w, or from 1 to 3% w/w, or from 1 to 2% w/w. In specific embodiments, the magnesium stearate is present in an amount of 0.1%, or 0.5, or 0.7%, or 0.9%, or 1.0%, or 1.2% w/w. In a further specific embodiment, the magnesium stearate is in an amount of 1% w/w.

In one embodiment, the pharmaceutical composition comprises a) about 1 to about 10% w/w of obicetrapib, or a salt, solvate or hydrate thereof, and b) about 5 to about 20% w/w of the SGLT2 inhibitor, or a salt, solvate or hydrate thereof. In a related embodiment, the composition comprises a) about 5% w/w of obicetrapib, or a salt, solvate or hydrate thereof, and b) about 5-20% w/w of the SGLT2 inhibitor, or a salt, solvate or hydrate thereof. In another embodiment, the composition comprises a) about 10% w/w of obicetrapib, or a salt, solvate or hydrate thereof, and b) about 5-25% w/w of the SGLT2 inhibitor, or a salt, solvate or hydrate thereof. In yet a further related embodiment, the composition further comprises a) about 40 to about 65% w/w microcrystalline cellulose, b) about 5 to about 23% w/w mannitol, c) about 1 to about 10% w/w sodium starch glycolate, d) about 0.5 to about 3% w/w colloidal silicon dioxide, and e) about 0.1 to about 3% w/w magnesium stearate. In another embodiment, the composition comprises a) about 10% w/w of obicetrapib, or a salt, solvate or hydrate thereof, and b) about 5-25% w/w of the SGLT2 inhibitor, or a salt, solvate or hydrate thereof, c) about 40 to about 65% w/w microcrystalline cellulose, d) about 5 to about 23% w/w mannitol, e) about 1 to about 10% w/w sodium starch glycolate, f) about 0.5 to about 3% w/w colloidal silicon dioxide, and g) about 0.1 to about 3% w/w magnesium stearate.

In one embodiment, the pharmaceutical composition comprises a) 1 to 10% w/w of obicetrapib, or a salt, solvate or hydrate thereof, and b) 5 to 20% w/w of the SGLT2 inhibitor, or a salt, solvate or hydrate thereof. In a related embodiment, the composition comprises a) 5% w/w of obicetrapib, or a salt, solvate or hydrate thereof, and b) 5-20% w/w of the SGLT2 inhibitor, or a salt, solvate or hydrate thereof. In another embodiment, the composition comprises a) 10% w/w of obicetrapib, or a salt, solvate or hydrate thereof, and b) 5-25% w/w of the SGLT2 inhibitor, or a salt, solvate or hydrate thereof. In yet a further related embodiment, the composition further comprises a) 40 to 65% w/w microcrystalline cellulose, b) 5 to 23% w/w mannitol, c) 1 to 10% w/w sodium starch glycolate, d) 0.5 to 3% w/w colloidal silicon dioxide, and e) 0.1 to 3% w/w magnesium stearate. In another embodiment, the composition comprises a) 10% w/w of obicetrapib, or a salt, solvate or hydrate thereof, and b) 5-25% w/w of the SGLT2 inhibitor, or a salt, solvate or hydrate thereof, c) 40 to 65% w/w microcrystalline cellulose, d) 5 to 23% w/w mannitol, e) 1 to 10% w/w sodium starch glycolate, f) 0.5 to 3% w/w colloidal silicon dioxide, and g) 0.1 to 3% w/w magnesium stearate.

In certain embodiments, the pharmaceutical composition comprises:

| | Amount (% by weight) |
|---|---|
| Obicetrapib | 1-10 |
| SGTL2 inhibitor | 4-75 |
| one or more diluents | 5-85 |
| one or more disintegrants | 1-15 |
| one or more additional additives | up to100% |

In certain embodiments, the pharmaceutical composition comprises:

| | Amount (% by weight) |
|---|---|
| Obicetrapib | 3-10 |
| SGTL2 inhibitor | 7-20 |
| one or more diluents | 65-85 |
| one or more disintegrants | 1-5 |
| one or more additional additives | up to100% |

In certain embodiments, the SGLT2 inhibitor is empagliflozin, in an amount of 7-9% w/w, or 18-20% w/w; and the obicetrapib is in an amount of 3.5-5% w/w, or 7-10% w/w.

In certain embodiments, the pharmaceutical composition comprises:

| | Amount (% by weight) |
|---|---|
| Obicetrapib | 4-10 |
| SGTL2 inhibitor | 4-10 |
| one or more diluents | 65-85 |
| one or more disintegrants | 1-5 |
| one or more additional additives | up to100% |

In certain embodiments, the SGLT2 inhibitor is dapagliflozin, in an amount of 4-5% w/w, or 8-10% w/w; and the obicetrapib is in an amount of 4-5% w/w, or 8-10% w/w.

In certain embodiments, the pharmaceutical composition comprises:

| | Amount (% by weight) |
|---|---|
| Obicetrapib | 4-10 |
| SGTL2 inhibitor | 4-15 |
| one or more diluents | 70-85 |
| one or more disintegrants | 1-5 |
| one or more additional additives | up to100% |

In certain embodiments, the SGLT2 inhibitor is ertugliflozin, in an amount of 4-5% w/w, or 11-13% w/w; and the obicetrapib is in an amount of 4-5% w/w, or 8-10% w/w.

In certain embodiments, the pharmaceutical composition comprises:

| | Amount (% by weight) |
|---|---|
| Obicetrapib | 1-5 |
| SGTL2 inhibitor | 40-75 |
| one or more diluents | 20-45 |
| one or more disintegrants | 1-5 |
| one or more additional additives | up to100% |

In certain embodiments, the SGLT2 inhibitor is canagliflozin, in an amount of 45-50% w/w, or 70-75% w/w; and the obicetrapib is in an amount of 1-3% w/w, or 3-5% w/w.

The pharmaceutical compositions described herein can be formulated with obicetrapib and an SGLT2 inhibitor as the two sole pharmaceutically active ingredients in the composition or can be combined with other active ingredients (e.g., as described herein).

In certain embodiments, the pharmaceutical composition is formulated into one or more suitable pharmaceutical preparations, such as solutions, suspensions, powders, sustained release formulations or elixirs in sterile solutions or suspensions for parenteral administration, or as transdermal patch preparation and dry powder inhalers.

In compositions provided herein, obicetrapib and the SGLT2 inhibitor described herein may be mixed with a suitable pharmaceutical carrier. The concentration of the each active in the compositions can, for example, be effective for delivery of an amount, upon administration, that treats, prevents, or ameliorates a condition or disorder described herein or a symptom thereof.

In certain embodiments, the pharmaceutical compositions provided herein are formulated for single dosage administration. To formulate a composition, the weight fraction of each active is dissolved, suspended, dispersed or otherwise mixed in a selected carrier at an effective concentration such that the treated condition is relieved, prevented, or one or more symptoms are ameliorated.

Concentrations of the obicetrapib and SGLT2 inhibitor in a pharmaceutical composition provided herein will depend on, e.g., the physicochemical characteristics of the compounds, the dosage schedule, and amount administered as well as other factors known to those of skill in the art. For example, if the composition comprises a salt of obicetrapib the amount of said salt to be administered and/or to be incorporated into a pharmaceutical composition (i.e., pharmaceutical dosage form) needs to be adjusted to take account of the molecular weight difference between the free base and salt form. For instance, in expressing dose amounts in the label and/or product information of authorized medicinal products comprising a salt form of an active compound that can also be used in free base form, it is customary practice to specify the dose of the free base to which the dose of the salt as used is equivalent.

Pharmaceutical compositions described herein are provided for administration to a subject, for example, humans or animals (e.g., mammals) in unit dosage forms, such as sterile parenteral (e.g., intravenous) solutions or suspensions containing suitable quantities of the compounds or pharmaceutically acceptable derivatives thereof. Pharmaceutical compositions are also provided for administration to humans and animals in unit dosage form, including oral or nasal solutions or suspensions and oil-water emulsions containing suitable quantities of a conjugate or pharmaceutically acceptable derivatives thereof. The conjugate is, in certain embodiments, formulated and administered in unit-dosage forms or multiple-dosage forms. Unit-dose forms as used herein refers to physically discrete units suitable for human or animal (e.g., mammal) subjects and packaged individually as is known in the art. Each unit-dose contains a predetermined quantity of obicetrapib and SGLT2 inhibitor sufficient to produce the desired therapeutic effect, in association with the required pharmaceutical carrier, vehicle or diluent. Examples of unit-dose forms include ampoules and syringes and individually packaged tablets. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of capsules or bottles. Hence, in specific aspects, multiple dose form is a multiple of unit-doses which are not segregated in packaging.

In certain embodiments, the obicetrapib and SGLT2 inhibitor described herein are in a liquid pharmaceutical formulation. Liquid pharmaceutically administrable formulations can, for example, be prepared by dissolving, dispersing, or otherwise mixing the active compounds and optional pharmaceutical adjuvants in a carrier, such as, for example, water, saline, aqueous dextrose, glycerol, glycols, and the like, to thereby form a solution or suspension. In certain embodiments, a pharmaceutical composition provided herein to be administered can also contain minor amounts of nontoxic auxiliary substances such as wetting agents, emulsifying agents, solubilizing agents, and pH buffering agents and the like.

Actual methods of preparing such dosage forms are known, or will be apparent, to those skilled in this art; for example, see, e.g., Remington: The Science and Practice of Pharmacy (Remington: The Science and Practice of Pharmacy, 23rd Edition, ISBN-13:978-0128200070) Dosage forms or compositions containing obicetrapib and an SGLT2 inhibitor in the ranges disclosed herein with the balance made up from non-toxic carrier can be prepared.

Parenteral administration, in certain embodiments, is characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. Other routes of administration may include, enteric administration, intracerebral administration, nasal administration, intraarterial administration, intracardiac administration, intraosseous infusion, intrathecal administration, and intraperitoneal administration.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Pharmaceutical carriers also include ethyl alcohol, polyethylene glycol and propylene glycol for water miscible vehicles; and sodium hydroxide, hydrochloric acid, citric acid or lactic acid for pH adjustment.

In certain embodiments, intravenous or intraarterial infusion of a sterile aqueous solution containing the combination described herein is an effective mode of administration. Another embodiment is a sterile aqueous or oily solution or suspension containing a conjugate described herein injected as necessary to produce the desired pharmacological effect.

In certain embodiments, the pharmaceutical formulations are lyophilized powders, which can be reconstituted for administration as solutions, emulsions and other mixtures. They can also be reconstituted and formulated as solids or gels.

The lyophilized powder is prepared by dissolving a compound provided herein, in a suitable solvent. In some embodiments, the lyophilized powder is sterile. Suitable solvents can contain an excipient which improves the stability or other pharmacological component of the powder or reconstituted solution, prepared from the powder. Excipients that can be used include, but are not limited to, dextrose, sorbitol, fructose, corn syrup, xylitol, glycerin, glucose, sucrose or other suitable agent. A suitable solvent can also contain a buffer, such as citrate, sodium or potassium phosphate or other such buffer known to those of skill in the art at, in certain embodiments, about neutral pH. Subsequent sterile filtration of the solution followed by lyophilization under standard conditions known to those of skill in the art provides an example of a formulation. In certain embodiments, the resulting solution will be apportioned into vials for lyophilization. Lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier.

In certain embodiments, the pharmaceutical composition is formulated in as a solid dosage form, such as a tablet or a capsule (e.g., as described herein below).

### 4.2. Pharmaceutical Dosage Forms

As summarized above, this disclosure provides a pharmaceutical dosage form comprising the pharmaceutical composition described herein. The disclosure provides for tablets, pills, and the like, comprising the pharmaceutical compositions or dosage forms described herein. The tablets or pills of the present disclosure may be coated to provide a dosage form affording the advantage of prolonged action or to protect from the acid conditions of the stomach. The tablets may also be formulated for immediate release as previously described. In certain embodiments, the tablet comprises a film coating. A film coating may be useful for limiting photolytic degradation. Suitable film coatings are selected by routine screening of commercially available preparations. In one embodiment, the film coating is a hypromellose-based coating. In certain embodiments, the coating represents 2-5% by weight of the total tablet composition and comprises a film-forming agent, a plasticizer, a glidant and optionally one or more pigments. In certain cases, the coating represents 3% of the total tablet composition. An exemplary film coating composition may comprise hydroxypropyl methylcellulose (HPMC), lactose monohydrate, titanium dioxide, and triglyceride 1,2,3-triacetoxypropane (triacetin). In certain cases, the film coating composition may comprise hydroxypropyl methylcellulose (HPMC), polyethylene glycol (PEG), talc, titanium dioxide and optionally iron oxide, including iron oxide red and/or yellow.

The tablets may be formulated into a monolayer or bilayer tablet. Typically, monolayer tablets comprise the active ingredients (i.e., obicetrapib and the SGLT2 inhibitor) co-mixed in a single uniform layer. For making monolayer tablets, exemplary methods include direct compression, wet granulation and dry granulation. The direct compression tablet process uses two primary process steps: blending the active ingredients with the excipients and compressing the finished tablet.

In one embodiment, the present disclosure provides a wet granulation process for making the subject pharmaceutical dosage form, wherein said process comprises the steps of:
(1) Premixing the active ingredient and the main portion of the excipients including the binder in a mixer to obtain a pre-mixture;
(2) granulating the pre-mixture of step (1) by adding the granulation liquid, e.g., purified water;
(3) drying the granules of step (2) in a fluidized bed dryer or a drying oven;
(4) optionally dry sieving of the dried granules of step (3);
(5) mixing the dried granules of step (4) with the remaining excipients like glidant and lubricant in a mixer to obtain the final mixture;
(6) tableting the final mixture of step (5) by compressing it on a suitable tablet press to produce tablets cores;
(7) optionally film-coating of the tablet cores of step (6) with a non-functional coat.

In certain embodiments, the present invention provides a pharmaceutical dosage form (e.g., as described herein) obtainable by a wet granulation process.

In another embodiment, the present disclosure provides a direct compression process for making the subject pharmaceutical dosage form, wherein said process comprises the steps of:
(1) Premixing the active ingredient and the main portion of the excipients in a mixer to obtain a pre-mixture;
(2) optionally dry screening the pre-mixture through a screen in order to segregate cohesive particles and to improve content uniformity;
(3) mixing the pre-mixture of step (1) or (2) in a mixer, optionally by adding remaining excipients to the mixture and continuing mixing;
(4) tableting the final mixture of step (3) by compressing it on a suitable tablet press to produce the tablet cores;
(5) optionally film-coating of the tablet cores of step (4) with a non-functional coat.

In certain embodiments, the present invention provides a pharmaceutical dosage form obtainable (e.g., as described herein) by a direct compression process.

In another embodiment, the present disclosure provides a dry granulation process for making the subject pharmaceutical dosage form, wherein said process comprises the steps of:
(1) mixing the active ingredient with either all or a portion of the excipients in a mixer;
(2) compaction of the mixture of step (1) on a suitable roller compactor;
(3) reducing the ribbons obtained during step (2) to granules, preferably small granules, by suitable milling or sieving steps;
(4) optionally mixing the granules of step (3) with the remaining excipients in a mixer to obtain the final mixture;
(5) tableting the granules of step (3) or the final mixture of step (4) by compressing it on a suitable tablet press to produce the tablet cores;
(6) optionally film-coating of the tablet cores of step (5) with a non-functional coat.

In certain embodiments, the present invention provides a pharmaceutical dosage form (e.g., as described herein) obtainable by a dry granulation process.

Bilayer tablets comprise the active ingredients (i.e., obicetrapib and the SGLT2 inhibitor) in separate layers and can be made by making a blend comprising excipients and one active ingredient (i.e., obicetrapib), and making a separate blend comprising the second active ingredient (i.e., the SGLT2 inhibitor) and excipients. One blend may then be pre-compressed, and the second blend may then be added on top of the first pre-compressed blends. The resulting tablet comprises two separate layers, each layer comprising a different active ingredient.

In certain embodiments, the pharmaceutical dosage form comprises the obicetrapib in a therapeutically effective amount (e.g., as described herein for obicetrapib).

In certain embodiments, the pharmaceutical dosage form comprises from about 1% to about 25% w/w of obicetrapib, or a salt, solvate or hydrate thereof. In further embodiments, the pharmaceutical dosage form comprises from about 1% to about 20% w/w, or from about 1% to about 15% w/w, or from about 1% to about 10% w/w, or from about 5% to about 15% w/w, or from about 5% to about 12% w/w of obicetrapib, or a salt, solvate or hydrate thereof. In further embodiments, the pharmaceutical dosage form comprises about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, or about 15% w/w of obicetrapib, or a salt, solvate or hydrate thereof. In a specific embodiment, the pharmaceutical dosage form comprises about 3-5% w/w of obicetrapib, or a salt, solvate or hydrate thereof. In a specific embodiment, the pharmaceutical dosage form comprises about 7-10% w/w of obicetrapib, or a salt, solvate or hydrate thereof.

In certain embodiments, the pharmaceutical dosage form comprises from 1% to 25% w/w of obicetrapib, or a salt, solvate or hydrate thereof. In further embodiments, the pharmaceutical dosage form comprises from 1% to 20% w/w, or from 1% to 15% w/w, or from 1% to 10% w/w, or from 5% to 15% w/w, or from 5% to 12% w/w of obicetrapib, or a salt, solvate or hydrate thereof. In further embodiments, the pharmaceutical dosage form comprises 1% w/w, 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w, or 15% w/w of obicetrapib, or a salt, solvate or hydrate thereof. In a specific embodiment, the pharmaceutical dosage form comprises 3-5% w/w of obicetrapib, or a salt, solvate or hydrate thereof. In a specific embodiment, the pharmaceutical dosage form comprises 7-10% w/w of obicetrapib, or a salt, solvate or hydrate thereof.

In some embodiments, the pharmaceutical dosage form comprises from about 1 mg to about 25 mg of obicetrapib, or a salt, solvate, or hydrate of obicetrapib in an equivalent dose. In further embodiments, the pharmaceutical dosage form comprises from about 1 mg to about 20 mg of obicetrapib, or from about 1 mg to about 15 mg of obicetrapib, or from about 1 mg to about 10 mg of obicetrapib, or from about 5 mg to about 15 mg of obicetrapib, or from about 5 mg to about 12 mg of obicetrapib, or a salt, solvate, or hydrate of obicetrapib in an equivalent dose. In further embodiments, the pharmaceutical dosage form comprises about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, or about 15 mg of obicetrapib, or a salt, solvate, or hydrate of obicetrapib in an equivalent dose. In a specific embodiment, the pharmaceutical dosage form comprises about 5 mg of obicetrapib, or a salt, solvate, or hydrate of obicetrapib in an equivalent dose. In a specific embodiment, the pharmaceutical dosage form comprises about 10 mg of obicetrapib, or a salt, solvate, or hydrate of obicetrapib in an equivalent dose. In another embodiment, the pharmaceutical dosage form comprises 5 mg of obicetrapib as the calcium salt. In a specific embodiment, the pharmaceutical dosage form comprises 10 mg of obicetrapib as the calcium salt.

In some embodiments, the pharmaceutical dosage form comprises from 1 mg to 25 mg of obicetrapib, or a salt, solvate, or hydrate of obicetrapib in an equivalent dose. In further embodiments, the pharmaceutical dosage form comprises from 1 mg to 20 mg of obicetrapib, or from 1 mg to 15 mg of obicetrapib, or from 1 mg to 10 mg of obicetrapib, or from 5 mg to 15 mg of obicetrapib, or from 5 mg to 12 mg of obicetrapib, or a salt, solvate, or hydrate of obicetrapib in an equivalent dose. In further embodiments, the pharmaceutical dosage form comprises 1 mg, 2 mg, 3 mg, 4 mg, 5 mg, 6 mg, 7 mg, 8 mg, 9 mg, 10 mg, 11 mg, 12 mg, 13 mg, 14 mg, or 15 mg of obicetrapib, or a salt, solvate, or hydrate of obicetrapib in an equivalent dose. In a specific embodiment, the pharmaceutical dosage form comprises 5 mg of obicetrapib, or a salt, solvate, or hydrate of obicetrapib in an equivalent dose. In a specific embodiment, the pharmaceutical dosage form comprises 10 mg of obicetrapib, or a salt, solvate, or hydrate of obicetrapib in an equivalent dose. In another embodiment, the pharmaceutical dosage form comprises 5 mg of obicetrapib as the calcium salt. In a specific embodiment, the pharmaceutical dosage form comprises 10 mg of obicetrapib as the calcium salt.

In certain embodiments, the pharmaceutical dosage form comprises the SGLT2 inhibitor in a therapeutically effective amount (e.g., as described herein for the subject SGLT2 inhibitors).

In some embodiments, the pharmaceutical dosage form comprises from about 1% to about 75% w/w of the SGLT2 inhibitor, or a salt, solvate or hydrate thereof. In further embodiments, the pharmaceutical dosage form comprises from about 1% to about 40% w/w, or from about 1% to about 30% w/w, about 1% to about 20% w/w, about 1% to about 10%, or from about 5% to about 15% w/w, or from about 10% to about 25% w/w, or from about 45% to about 75% w/w of the SGLT2 inhibitor, or a salt, solvate or hydrate thereof. In further embodiments, the pharmaceutical dosage form comprises about 5% w/w, about 10% w/w, about 15% w/w, about 20% w/w, about 25% w/w, about 30% w/w, about 35% w/w, about 40% w/w, about 45% w/w, about 50% w/w, about 55% w/w, about 60% w/w, about 65% w/w, about 70% w/w, or about 75% w/w, of the SGLT2 inhibitor, or a salt, solvate or hydrate thereof. In a specific embodiment, the pharmaceutical dosage form comprises about 5% w/w of the SGLT2 inhibitor, or a salt, solvate or hydrate thereof. In a specific embodiment, the pharmaceutical dosage form comprises about 10% w/w of the SGLT2 inhibitor, or a salt, solvate or hydrate thereof. In a specific embodiment, the pharmaceutical dosage form comprises about 15% w/w of the SGLT2 inhibitor, or a salt, solvate or hydrate thereof. In a specific embodiment, the pharmaceutical dosage form comprises about 25% w/w of the SGLT2 inhibitor, or a salt, solvate or hydrate thereof. In a specific embodiment, the pharmaceutical dosage form comprises about 50% w/w of the SGLT2 inhibitor, or a salt, solvate or hydrate thereof. In a specific embodiment, the pharmaceutical dosage form comprises about 75% w/w of the SGLT2 inhibitor, or a salt, solvate or hydrate thereof.

In some embodiments, the pharmaceutical dosage form comprises from 1% to 75% w/w of the SGLT2 inhibitor, or a salt, solvate or hydrate thereof. In further embodiments, the pharmaceutical dosage form comprises from 1% to 40% w/w, or from 1% to 30% w/w, 1% to 20% w/w, 1% to 10%, or from 5% to 15% w/w, or from 10% to 25% w/w, or from 45% to 75% w/w of the SGLT2 inhibitor, or a salt, solvate or hydrate thereof. In further embodiments, the pharmaceutical dosage form comprises 5% w/w, 10% w/w, 15% w/w, 20% w/w, 25% w/w, 30% w/w, 35% w/w, 40% w/w, 45% w/w, 50% w/w, 55% w/w, 60% w/w, 65% w/w, 70% w/w, or 75% w/w, of the SGLT2 inhibitor, or a salt, solvate or hydrate thereof. In a specific embodiment, the pharmaceutical dosage form comprises 5% w/w of the SGLT2 inhibitor, or a salt, solvate or hydrate thereof. In a specific embodiment, the pharmaceutical dosage form comprises 10% w/w of the SGLT2 inhibitor, or a salt, solvate or hydrate thereof. In a specific embodiment, the pharmaceutical dosage form comprises 15% w/w of the SGLT2 inhibitor, or a salt, solvate or hydrate thereof. In a specific embodiment, the pharmaceutical dosage form comprises 25% w/w of the SGLT2 inhibitor, or a salt, solvate or hydrate thereof. In a specific embodiment, the pharmaceutical dosage form comprises 50% w/w of the SGLT2 inhibitor, or a salt, solvate or hydrate thereof. In a specific embodiment, the pharmaceutical dosage form comprises 75% w/w of the SGLT2 inhibitor, or a salt, solvate or hydrate thereof.

In some embodiments, the pharmaceutical dosage form comprises from about 1 mg to about 300 mg of the SGLT2 inhibitor, or a salt, solvate, or hydrate of the SGLT2 inhibitor in an equivalent dose. In further embodiments, the pharmaceutical dosage form comprises from about 5 mg to about 100 mg of the SGLT2 inhibitor, or from about 5 mg to about 50 mg of the SGLT2 inhibitor, or from about 10 mg to about 25 mg of the SGLT2 inhibitor, or from about 5 mg to about 15 mg of the SGLT2 inhibitor, or from about 5 mg to about 10 mg of SGLT2 inhibitor, or a salt, solvate, or hydrate of the SGLT2 inhibitor in an equivalent dose. In further embodiments, the pharmaceutical composition comprises about 100 mg, about 300 mg, about 150 mg to 250 mg, or about 100 mg to 200 mg of the SGLT2 inhibitor, or a salt, solvate, or hydrate of the SGLT2 inhibitor in an equivalent dose. In certain embodiments, the pharmaceutical dosage form comprises about 5 mg, about 7 mg, about 8 mg, about 10 mg, about 12 mg, about 15 mg, about 18 mg, about 20 mg, about 22 mg, about 25 mg, about 30 mg, about 40 mg, or about 50 mg of the SGLT2 inhibitor, or a salt, solvate, or hydrate of the SGLT2 inhibitor in an equivalent dose. In a specific embodiment, the pharmaceutical dosage form comprises about 5 mg of the SGLT2 inhibitor, or a salt, solvate, or hydrate of the SGLT2 inhibitor in an equivalent dose. In a specific embodiment, the pharmaceutical dosage form comprises about 10 mg of the SGLT2 inhibitor, or a salt, solvate, or hydrate of the SGLT2 inhibitor in an equivalent dose. In a specific embodiment, the pharmaceutical dosage form comprises about 15 mg of the SGLT2 inhibitor, or a salt, solvate, or hydrate of the SGLT2 inhibitor in an equivalent dose. In a specific embodiment, the pharmaceutical dosage form comprises about 25 mg of the SGLT2 inhibitor, or a salt, solvate, or hydrate of the SGLT2 inhibitor in an equivalent dose. In a specific embodiment, the pharmaceutical dosage form comprises about 100 mg of the SGLT2 inhibitor, or a salt, solvate, or hydrate of the SGLT2 inhibitor in an equivalent dose. In a specific embodiment, the pharmaceutical dosage form comprises about 300 mg of the SGLT2 inhibitor, or a salt, solvate, or hydrate of the SGLT2 inhibitor in an equivalent dose. In certain embodiments, any of the above amounts are of the free base form of the SGLT2 inhibitor, or a salt, solvate, or hydrate of the SGLT2 inhibitor in an equivalent dose. In certain some embodiments, the amounts are of a salt form of the SGLT2 inhibitor, or a salt, solvate, or hydrate of the SGLT2 inhibitor in an equivalent dose.

In some embodiments, the pharmaceutical dosage form comprises from 1 mg to 300 mg of the SGLT2 inhibitor, or a salt, solvate, or hydrate of the SGLT2 inhibitor in an equivalent dose. In further embodiments, the pharmaceutical dosage form comprises from 5 mg to 100 mg of the SGLT2 inhibitor, or from 5 mg to 50 mg of the SGLT2 inhibitor, or from 10 mg to 25 mg of the SGLT2 inhibitor, or from 5 mg to 15 mg of the SGLT2 inhibitor, or from 5 mg to 10 mg of SGLT2 inhibitor, or a salt, solvate, or hydrate of the SGLT2 inhibitor in an equivalent dose. In further embodiments, the pharmaceutical composition comprises 100 mg, 300 mg, 150 mg to 250 mg, or 100 mg to 200 mg of the SGLT2 inhibitor, or a salt, solvate, or hydrate of the SGLT2 inhibitor in an equivalent dose. In certain embodiments, the pharmaceutical dosage form comprises 5 mg, 7 mg, 8 mg, 10 mg, 12 mg, 15 mg, 18 mg, 20 mg, 22 mg, 25 mg, 30 mg, 40 mg, or 50 mg of the SGLT2 inhibitor, or a salt, solvate, or hydrate of the SGLT2 inhibitor in an equivalent dose. In a specific embodiment, the pharmaceutical dosage form comprises 5 mg of the SGLT2 inhibitor, or a salt, solvate, or hydrate of the SGLT2 inhibitor in an equivalent dose. In a specific embodiment, the pharmaceutical dosage form comprises 10 mg of the SGLT2 inhibitor, or a salt, solvate, or hydrate of the SGLT2 inhibitor in an equivalent dose. In a specific embodiment, the pharmaceutical dosage form comprises 15 mg of the SGLT2 inhibitor, or a salt, solvate, or hydrate of the SGLT2 inhibitor in an equivalent dose. In a specific embodiment, the pharmaceutical dosage form comprises 25 mg of the SGLT2 inhibitor, or a salt, solvate, or hydrate of the SGLT2 inhibitor in an equivalent dose. In a specific embodiment, the pharmaceutical dosage form comprises 100 mg of the SGLT2 inhibitor, or a salt, solvate, or hydrate of the SGLT2 inhibitor in an equivalent dose. In a specific embodiment, the pharmaceutical dosage form comprises 300 mg of the SGLT2 inhibitor, or a salt, solvate, or hydrate of the SGLT2 inhibitor in an equivalent dose. In certain embodiments, any of the above amounts are of the free base form of the SGLT2 inhibitor, or a salt, solvate, or hydrate of the SGLT2 inhibitor in an equivalent dose. In certain some embodiments, the amounts are of a salt form of the SGLT2 inhibitor, or a salt, solvate, or hydrate of the SGLT2 inhibitor in an equivalent dose.

In one embodiment, the pharmaceutical dosage form comprises a) about 5 mg to about 10 mg of obicetrapib, or a salt, solvate, or hydrate of obicetrapib in an equivalent dose and, b) about 10 mg to about 25 mg of the SGLT2 inhibitor, or a salt, solvate, or hydrate of the SGLT2 inhibitor in an equivalent dose. In a related embodiment, the dosage form comprises a) about 5 mg of obicetrapib, or a salt, solvate, or hydrate of obicetrapib in an equivalent dose, and b) about 10 mg of the SGLT2 inhibitor, or a salt, solvate, or hydrate of the SGLT2 inhibitor in an equivalent dose. In a related embodiment, the dosage form comprises a) about 5 mg of obicetrapib, or a salt, solvate, or hydrate of obicetrapib in an equivalent dose, and b) about 25 mg of the SGLT2 inhibitor, or a salt, solvate, or hydrate of the SGLT2 inhibitor in an equivalent dose. In another embodiment, the tablet comprises a) about 10 mg of obicetrapib, or a salt, solvate, or hydrate of obicetrapib in an equivalent dose, and b) about 10 mg of the SGLT2 inhibitor, or a salt, solvate, or hydrate of the SGLT2 inhibitor in an equivalent dose. In a related embodiment, the dosage form comprises a) about 10 mg of obicetrapib, or a salt, solvate, or hydrate of obicetrapib in an equivalent dose, and b) about 25 mg of the SGLT2 inhibitor, or a salt, solvate, or hydrate of the SGLT2 inhibitor in an equivalent dose. In certain embodiments, the SGLT2 inhibitor is empagliflozin.

In one embodiment, the pharmaceutical dosage form comprises a) 5 mg to 10 mg of obicetrapib, or a salt, solvate, or hydrate of obicetrapib in an equivalent dose and, b) 10 mg to 25 mg of the SGLT2 inhibitor, or a salt, solvate, or hydrate of the SGLT2 inhibitor in an equivalent dose. In a related embodiment, the dosage form comprises a) 5 mg of obicetrapib, or a salt, solvate, or hydrate of obicetrapib in an equivalent dose, and b) 10 mg of the SGLT2 inhibitor, or a salt, solvate, or hydrate of the SGLT2 inhibitor in an equivalent dose. In a related embodiment, the dosage form comprises a) 5 mg of obicetrapib, or a salt, solvate, or hydrate of obicetrapib in an equivalent dose, and b) 25 mg of the SGLT2 inhibitor, or a salt, solvate, or hydrate of the SGLT2 inhibitor in an equivalent dose. In another embodiment, the tablet comprises a) 10 mg of obicetrapib, or a salt, solvate, or hydrate of obicetrapib in an equivalent dose, and b) 10 mg of the SGLT2 inhibitor, or a salt, solvate, or hydrate of the SGLT2 inhibitor in an equivalent dose. In a related embodiment, the dosage form comprises a) 10 mg of obicetrapib, or a salt, solvate, or hydrate of obicetrapib in an equivalent dose, and b) 25 mg of the SGLT2 inhibitor, or a salt, solvate, or hydrate of the SGLT2 inhibitor in an equivalent dose. In certain embodiments, the SGLT2 inhibitor is empagliflozin.

In another embodiment, the pharmaceutical dosage form comprises a) about 5 mg to about 10 mg of obicetrapib, or a salt, solvate, or hydrate of obicetrapib in an equivalent dose, and b) about 5 mg to about 15 mg of the SGLT2 inhibitor, or a salt, solvate, or hydrate of the SGLT2 inhibitor in an equivalent dose. In a related embodiment, the dosage form comprises a) about 5 mg of obicetrapib, or a salt, solvate, or hydrate of obicetrapib in an equivalent dose, and b) about 5 mg of the SGLT2 inhibitor, or a salt, solvate, or hydrate of the SGLT2 inhibitor in an equivalent dose. In a related embodiment, the dosage form comprises a) about 5 mg of obicetrapib, or a salt, solvate, or hydrate of obicetrapib in an equivalent dose, and b) about 15 mg of the SGLT2 inhibitor, or a salt, solvate, or hydrate of the SGLT2 inhibitor in an equivalent dose. In another embodiment, the tablet comprises a) about 10 mg of obicetrapib, or a salt, solvate, or hydrate of obicetrapib in an equivalent dose, and b) about 5 mg of the SGLT2 inhibitor, or a salt, solvate, or hydrate of the SGLT2 inhibitor in an equivalent dose. In another embodiment, the tablet comprises a) about 10 mg of obicetrapib, or a salt, solvate, or hydrate of obicetrapib in an equivalent dose, and b) about 15 mg of the SGLT2 inhibitor, or a salt, solvate, or hydrate of the SGLT2 inhibitor in an equivalent dose. In certain embodiments, the SGLT2 inhibitor is ertugliflozin.

In another embodiment, the pharmaceutical dosage form comprises a) 5 mg to 10 mg of obicetrapib, or a salt, solvate, or hydrate of obicetrapib in an equivalent dose, and b) 5 mg to 15 mg of the SGLT2 inhibitor, or a salt, solvate, or hydrate of the SGLT2 inhibitor in an equivalent dose. In a related embodiment, the dosage form comprises a) 5 mg of obicetrapib, or a salt, solvate, or hydrate of obicetrapib in an equivalent dose, and b) 5 mg of the SGLT2 inhibitor, or a salt, solvate, or hydrate of the SGLT2 inhibitor in an equivalent dose. In a related embodiment, the dosage form comprises a) 5 mg of obicetrapib, or a salt, solvate, or hydrate of obicetrapib in an equivalent dose, and b) 15 mg of the SGLT2 inhibitor, or a salt, solvate, or hydrate of the SGLT2 inhibitor in an equivalent dose. In another embodiment, the tablet comprises a) 10 mg of obicetrapib, or a salt, solvate, or hydrate of obicetrapib in an equivalent dose, and b) 5 mg of the SGLT2 inhibitor, or a salt, solvate, or hydrate of the SGLT2 inhibitor in an equivalent dose. In another embodiment, the tablet comprises a) 10 mg of obicetrapib, or a salt, solvate, or hydrate of obicetrapib in an equivalent dose, and b) 15 mg of the SGLT2 inhibitor, or a salt, solvate, or hydrate of the SGLT2 inhibitor in an equivalent dose. In certain embodiments, the SGLT2 inhibitor is ertugliflozin.

In another embodiment, the pharmaceutical dosage form comprises a) about 5 mg to about 10 mg of obicetrapib, or a salt, solvate, or hydrate of obicetrapib in an equivalent dose, and b) about 5 mg to about 10 mg of the SGLT2 inhibitor, or a salt, solvate, or hydrate of the SGLT2 inhibitor in an equivalent dose. In certain embodiments, the SGLT2 inhibitor is dapagliflozin.

In another embodiment, the pharmaceutical dosage form comprises a) 5 mg to 10 mg of obicetrapib, or a salt, solvate, or hydrate of obicetrapib in an equivalent dose, and b) 5 mg to 10 mg of the SGLT2 inhibitor, or a salt, solvate, or hydrate of the SGLT2 inhibitor in an equivalent dose. In certain embodiments, the SGLT2 inhibitor is dapagliflozin.

In another embodiment, the pharmaceutical dosage form comprises a) about 5 mg to about 10 mg of obicetrapib, or a salt, solvate, or hydrate of obicetrapib in an equivalent dose, and b) about 100 mg to about 300 mg of the SGLT2 inhibitor, or a salt, solvate, or hydrate of the SGLT2 inhibitor in an equivalent dose. In a related embodiment, the dosage form comprises a) about 5 mg of obicetrapib, or a salt, solvate, or hydrate of obicetrapib in an equivalent dose, and b) about 100 mg of the SGLT2 inhibitor, or a salt, solvate, or hydrate of the SGLT2 inhibitor in an equivalent dose. In a related embodiment, the dosage form comprises a) about 5 mg of obicetrapib, or a salt, solvate, or hydrate of obicetrapib in an equivalent dose, and b) about 300 mg of the SGLT2 inhibitor, or a salt, solvate, or hydrate of the SGLT2 inhibitor in an equivalent dose. In a related embodiment, the dosage form comprises a) about 10 mg of obicetrapib, or a salt, solvate, or hydrate of obicetrapib in an equivalent dose, and b) about 100 mg of the SGLT2 inhibitor, or a salt, solvate, or hydrate of the SGLT2 inhibitor in an equivalent dose. In another embodiment, the tablet comprises a) about 10 mg of obicetrapib, or a salt, solvate, or hydrate of obicetrapib in an equivalent dose and b) about 300 mg of the SGLT2 inhibitor, or a salt, solvate, or hydrate of the SGLT2 inhibitor in an equivalent dose. In certain embodiments, the SGLT2 inhibitor is canagliflozin.

In another embodiment, the pharmaceutical dosage form comprises a) 5 mg to 10 mg of obicetrapib, or a salt, solvate, or hydrate of obicetrapib in an equivalent dose, and b) 100 mg to 300 mg of the SGLT2 inhibitor, or a salt, solvate, or hydrate of the SGLT2 inhibitor in an equivalent dose. In a related embodiment, the dosage form comprises a) 5 mg of obicetrapib, or a salt, solvate, or hydrate of obicetrapib in an equivalent dose, and b) 100 mg of the SGLT2 inhibitor, or a salt, solvate, or hydrate of the SGLT2 inhibitor in an equivalent dose. In a related embodiment, the dosage form comprises a) 5 mg of obicetrapib, or a salt, solvate, or hydrate of obicetrapib in an equivalent dose, and b) 300 mg of the SGLT2 inhibitor, or a salt, solvate, or hydrate of the SGLT2 inhibitor in an equivalent dose. In a related embodiment, the dosage form comprises a) 10 mg of obicetrapib, or a salt, solvate, or hydrate of obicetrapib in an equivalent dose, and b) 100 mg of the SGLT2 inhibitor, or a salt, solvate, or hydrate of the SGLT2 inhibitor in an equivalent dose. In another embodiment, the tablet comprises a) 10 mg of obicetrapib, or a salt, solvate, or hydrate of obicetrapib in an equivalent dose and b) 300 mg of the SGLT2 inhibitor, or a salt, solvate, or hydrate of the SGLT2 inhibitor in an equivalent dose. In certain embodiments, the SGLT2 inhibitor is canagliflozin.

As used herein the term “equivalent dose” refers to an amount of a given compound “equivalent” to a specified amount of a reference compound (e.g., a free base form of a compound). For example, if the dosage form comprises the salt of obicetrapib, the amount of the salt to be incorporated in the pharmaceutical dosage form needs to be adjusted to take account of the molecular weight difference between obicetrapib as the free base form and the salt form.

In certain embodiments, the pharmaceutical dosage form comprises one or more additional active compounds (e.g., as described herein). In certain embodiments, the pharmaceutical dosage form further comprises a therapeutically effective amount of metformin (e.g., metformin hydrochloride). In certain embodiments, the pharmaceutical dosage form comprises from 500 mg to 1000 mg of metformin, such as 500 mg to 900 mg, 500 mg to 800 mg, 500 mg to 700 mg, or 500 mg to 600 mg of metformin (e.g., metformin hydrochloride).

In certain embodiments, the pharmaceutical form further comprises a therapeutically effective amount of linagliptin. In certain embodiments, the pharmaceutical dosage form comprises from 1 mg to 10 mg of linagliptin, such as 1 mg to 9 mg, 1 mg to 8 mg, 1 mg to 7 mg, 1 mg to 6 mg, 1 mg to 5 mg, 2 mg to 5 mg, 2 mg to 4 mg, of linagliptin, or a salt, solvate, or hydrate of linagliptin in an equivalent dose. In certain cases, the pharmaceutical composition comprises 2.5 mg of linagliptin, or a salt, solvate, or hydrate of linagliptin in an equivalent dose. In certain cases, the pharmaceutical composition comprises 5 mg of linagliptin, or a salt, solvate, or hydrate of linagliptin in an equivalent dose.

In certain embodiments, the pharmaceutical dosage form comprises one or more excipients (e.g., as described herein). In certain embodiments, the pharmaceutical dosage form comprises one or more diluents. In certain embodiments, the pharmaceutical dosage from comprises microcrystalline cellulose, mannitol, or a combination of both.

In certain embodiments, the pharmaceutical dosage form comprises microcrystalline cellulose in an amount from about 40 mg to 80 mg, such as 45 mg to 75 mg, 45 mg to 70 mg, 45 mg to 65 mg, or 50 to 65 mg. In a specific embodiment, the microcrystalline cellulose is in an amount of about 60 mg. In a further specific embodiment, the microcrystalline cellulose is in an amount of about 65 mg.

In certain embodiments, the pharmaceutical dosage form comprises microcrystalline cellulose in an amount from 40 mg to 80 mg, such as 45 mg to 75 mg, 45 mg to 70 mg, 45 mg to 65 mg, or 50 to 65 mg. In a specific embodiment, the microcrystalline cellulose is in an amount of 60 mg. In a further specific embodiment, the microcrystalline cellulose is in an amount of 65 mg.

In certain embodiments, the pharmaceutical dosage form comprises mannitol in an amount from about 1 mg to 35 mg, such as 1 mg to 30 mg, 1 mg to 30 mg, 1 mg to 25 mg, or 10 to 25 mg. In a specific embodiment, the microcrystalline cellulose is in an amount of about 23 mg.

In embodiments, the pharmaceutical dosage form comprises mannitol in an amount from 1 mg to 35 mg, such as 1 mg to 30 mg, 1 mg to 30 mg, 1 mg to 25 mg, or 10 to 25 mg. In a specific embodiment, the microcrystalline cellulose is in an amount of 23 mg.

In certain embodiments, the pharmaceutical dosage form comprises one or more disintegrants. In certain cases, the disintegrant is sodium starch glycolate. In certain embodiments, the pharmaceutical dosage form comprises sodium starch glycolate in an amount from about 1 mg to 15 mg, such as 1 mg to 10 mg, 1 mg to 8 mg, 2 mg to 7 mg, or 4 to 6 mg. In a specific embodiment, the sodium starch glycolate is in an amount of about 5 mg.

In certain embodiments, the pharmaceutical dosage form comprises sodium starch glycolate in an amount from 1 mg to 15 mg, such as 1 mg to 10 mg, 1 mg to 8 mg, 2 mg to 7 mg, or 4 to 6 mg. In a specific embodiment, the sodium starch glycolate is in an amount of 5 mg.

In certain embodiments, the pharmaceutical dosage form comprises one or more glidants. In certain cases, the glidant is colloidal silicon dioxide. In certain embodiments, the pharmaceutical dosage form comprises colloidal silicon dioxide in an amount from about 0.1 mg to 5 mg, such as 0.1 mg to 4 mg, 0.5 mg to 5 mg, 0.5 mg to 3 mg, or 0.5 to 2 mg. In a specific embodiment, the colloidal silicon dioxide is in an amount of about 1 mg.

In certain embodiments, the pharmaceutical dosage form comprises colloidal silicon dioxide in an amount from 0.1 mg to 5 mg, such as 0.1 mg to 4 mg, 0.5 mg to 5 mg, 0.5 mg to 3 mg, or 0.5 to 2 mg. In a specific embodiment, the colloidal silicon dioxide is in an amount of 1 mg.

In certain embodiments, the pharmaceutical dosage form comprises one or more lubricants. In certain cases, the lubricant is magnesium stearate. In certain embodiments, the pharmaceutical dosage form comprises magnesium stearate in an amount from about 0.1 mg to 5 mg, such as 0.1 mg to 4 mg, 0.5 mg to 5 mg, 0.5 mg to 3 mg, or 0.5 to 2 mg. In a specific embodiment, the colloidal silicon dioxide is in an amount of about 1 mg.

In certain embodiments, the pharmaceutical dosage form comprises magnesium stearate in an amount from 0.1 mg to 5 mg, such as 0.1 mg to 4 mg, 0.5 mg to 5 mg, 0.5 mg to 3 mg, or 0.5 to 2 mg. In a specific embodiment, the colloidal silicon dioxide is in an amount of 1 mg.

In further embodiments, the pharmaceutical composition, pharmaceutical dosage form, or tablet as described herein is free of negative drug-drug interactions. In a related embodiment, the pharmaceutical composition, pharmaceutical dosage form, or tablet is free of negative drug-drug interactions with other antidiabetic agents. In a further embodiment, the pharmaceutical composition, pharmaceutical dosage form, or tablet as described herein is administrable without regard to food and with or without regard to the patient being on another antidiabetic agent.

### 4.3. Methods of Use

As summarized above, also provided herein are methods of treating or preventing a metabolic disorder, the method comprising administering to a subject having or at risk of developing a metabolic disorder, a therapeutically effective amount of a pharmaceutical composition, or a pharmaceutical dosage form comprising a pharmaceutical composition, that comprises a combination of obicetrapib and an SGLT2 inhibitor, as described herein.

In another aspect, methods are provided for treating or preventing a metabolic disorder, the method comprising administering to a subject having or at risk of developing a metabolic disorder a therapeutically effective amount of a obicetrapib and administering a therapeutically effective amount of an SGLT2 inhibitor.

Without being bound to any particular theory, a pharmaceutical composition or dosage form comprising a pharmaceutical composition comprising a combination of obicetrapib and a SGLT2 inhibitor as defined herein, or separately administering both obicetrapib and an SGLT2 inhibitor, can be used for preventing, slowing progression of, delaying or treating a metabolic disorder, and in particular for delaying progression to insulin-dependence. This opens up new therapeutic possibilities in the treatment and prevention of type 2 diabetes mellitus, obesity, complications of diabetes mellitus and of neighboring disease states.

Accordingly, the present disclosure provides a method for preventing, slowing the progression of, delaying or treating a metabolic disorder selected from the group consisting of type 1 diabetes mellitus, type 2 diabetes mellitus, impaired glucose tolerance (IGT), impaired fasting blood glucose (IFG), hyperglycemia, postprandial hyperglycemia, obesity and metabolic syndrome in a patient in need thereof, by administering a therapeutically effective amount of a pharmaceutical composition or a pharmaceutical dosage form of the present disclosure to the patient, or separately administering both obicetrapib and an SGLT2 inhibitor.

The pharmaceutical composition according to the present disclosure, or the separate administration of both obicetrapib and an SGLT2 inhibitor, may also have valuable disease-modifying properties with respect to diseases or conditions related to impaired glucose tolerance (IGT), impaired fasting blood glucose (IFG), insulin resistance and/or metabolic syndrome.

Also provided herein is a method for preventing, slowing, delaying or reversing progression from impaired glucose tolerance (IGT), impaired fasting blood glucose (IFG), insulin resistance and/or from metabolic syndrome to type 2 diabetes mellitus in a patient in need thereof, by administering a therapeutically effective amount of a pharmaceutical composition or a pharmaceutical dosage form of the present disclosure to the patient, or by separately administering both obicetrapib and an SGLT2 inhibitor.

As such, the pharmaceutical compositions and dosage forms described herein can be effective to treat conditions and/or diseases related to or caused by an increased blood glucose level.

In various embodiments, there is provided a method for preventing, slowing the progression of, delaying or treating of a condition or disorder selected from the group consisting of complications of diabetes mellitus such as cataracts and micro- and macrovascular diseases, such as nephropathy, retinopathy, neuropathy, tissue ischemia, diabetic foot, arteriosclerosis, myocardial infarction, acute coronary syndrome, unstable angina pectoris, stable angina pectoris, stroke, peripheral arterial occlusive disease, cardiomyopathy, heart failure, heart rhythm disorders and vascular restenosis, by administering a therapeutically effective amount of a pharmaceutical composition or a pharmaceutical dosage form of the present disclosure to the patient, or by separately administering both obicetrapib and an SGLT2 inhibitor. In particular, one or more aspects of diabetic nephropathy such as hyperperfusion, proteinuria and albuminuria may be treated, their progression slowed or their onset delayed or prevented. The term “tissue ischemia” particularly comprises diabetic macroangiopathy, diabetic microangiopathy, impaired wound healing and diabetic ulcer.

By the administration of a pharmaceutical composition according to the present disclosure, or by separately administering both obicetrapib and an SGLT2 inhibitor, and due to the activity of the obicetrapib and SGLT2 inhibitor, excessive blood glucose levels may not be converted to insoluble storage forms, like fat, but excreted through the urine of the patient, and LDL cholesterol levels are reduced. As such, the pharmaceutical compositions and dosage forms described herein do not cause weight gain, and in some cases can even result in a reduction in body weight.

Accordingly, in some embodiments of the subject methods there is provided a method for reducing body weight or preventing an increase in body weight or facilitating a reduction in body weight in a patient in need thereof, by administering a therapeutically effective amount of a pharmaceutical composition or a pharmaceutical dosage form of the present disclosure to the patient, or by separately administering both obicetrapib and an SGLT2 inhibitor.

The pharmacological effect of the combination of obicetrapib and an SGLT2 inhibitor in the pharmaceutical composition, or through separate administration, according to the present disclosure is independent of insulin. Therefore, an improvement of the glycemic control is possible without an additional strain on the pancreatic beta cells, and an improvement in beta cell health and function is possible by reduction of lipid-induced toxicity. By an administration of a pharmaceutical composition according to the present disclosure, or separate administration of both obicetrapib and an SGLT2 inhibitor, beta-cell degeneration and a decline of beta-cell functionality, such as for example apoptosis or necrosis of pancreatic beta cells, can be delayed or prevented. Furthermore, the functionality of pancreatic cells can be improved or restored, and the number and size of pancreatic beta cells increased. It may be shown that the differentiation status and hyperplasia of pancreatic beta-cells disturbed by hyperglycemia and hyperlipidemia or dyslipidemia can be normalized by treatment with a pharmaceutical composition or dosage form according to the present disclosure.

Accordingly, also provided herein is a method for preventing, slowing, delaying or treating the degeneration of pancreatic beta cells and/or the decline of the functionality of pancreatic beta cells and/or for improving and/or restoring the functionality of pancreatic beta cells and/or restoring the functionality of pancreatic insulin secretion in a patient in need thereof, by administering a therapeutically effective amount of a pharmaceutical composition or a pharmaceutical dosage form of the present disclosure to the patient, or by separately administering both obicetrapib and an SGLT2 inhibitor.

By the administration of a pharmaceutical composition according to the present disclosure, or separate administration of both obicetrapib and an SGLT2 inhibitor, and due to the activity of the obicetrapib and SGLT2 inhibitor, an abnormal accumulation of fat in the liver may be reduced or inhibited. Therefore, according to present disclosure, there is also provided a method for preventing, slowing, delaying or treating diseases or conditions attributed to an abnormal accumulation of liver fat in a patient in need thereof, the method comprising administering a therapeutically effective amount of a pharmaceutical composition or a pharmaceutical dosage form of the present disclosure to the patient, or by separately administering both obicetrapib and an SGLT2 inhibitor. Diseases or conditions which are attributed to an abnormal accumulation of liver fat are particularly selected from the group consisting of general fatty liver, non-alcoholic fatty liver (NAFL), non-alcoholic steatohepatitis (NASH), hyperalimentation-induced fatty liver, diabetic fatty liver, alcoholic-induced fatty liver or toxic fatty liver.

As such, also provided herein is a method for maintaining and/or improving the insulin sensitivity and/or for treating or preventing hyperinsulinemia and/or insulin resistance in a patient in need thereof, by administering a therapeutically effective amount of a pharmaceutical composition or a pharmaceutical dosage form of the present disclosure to the patient, or by separately administering both obicetrapib and an SGLT2 inhibitor.

### 4.4. Kits

Also provided herein is a pharmaceutical kit comprising a package containing a plurality of unit pharmaceutical dosage forms (e.g., as described herein) and instructions for use.

In accordance with embodiments of the invention, the pharmaceutical kit comprises a container, such as a high-density polyethylene (HDPE) bottles, or a box including one or more blister packs, wherein the bottles or blister packs can contain a plurality of solid unit pharmaceutical dosage forms as described herein. In certain embodiments, the container or pack comprises at least 5, at least 8, at least 10, at least 12 of at least 15 of said unit pharmaceutical dosage forms, e.g. 4, 5, 6, 7, 8, 9, 10, 11, 12, 13, 14, 15, 16, 17, 18, 19 or 20 of said unit dosage forms.

In accordance with the invention, the pharmaceutical kit comprises instructions (e.g., a leaflet) inserted into the container or box, typically a patient information leaflet containing printed information, which information may include a description of the form and composition of the unit pharmaceutical dosage forms contained in the kit, an indication of the therapeutic indications for which the product is intended, instructions as to how the product is to be used and information and warnings concerning adverse effects and contraindications associated with the use. In accordance with the present disclosure, the leaflet will usually contain the information concerning the therapeutic indications, uses, treatment regimens, etc. as described herein in relation to the methods of treatment of the present invention. In certain cases, the leaflet contains printed instructions to repeatedly (self-) administer the pharmaceutical unit dosage forms in order to treat and/or prevent a metabolic disorder, in particular type 2 diabetes mellitus.

### 4.5. Definitions

The terms “subject” and “patient” are used interchangeably. A subject can be a mammal such as a non-primate (e.g., cows, pigs, horses, cats, dogs, goats, rabbits, rats, mice, etc.) or a primate (e.g., monkey and human), for example a human. In certain embodiments, the subject is a mammal, e.g., a human, diagnosed with a disease or disorder provided herein. In another embodiment, the subject is a mammal, e.g., a human, at risk of developing a disease or disorder provided herein. In a specific embodiment, the subject is human.

The terms “therapies” and “therapy” are used in their broadest sense understood in the clinical arts.

The term “pharmaceutically acceptable” indicates that the material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables.

The term “carrier” refers to a glidant, diluent, adjuvant, excipient, or vehicle etc. with which the compound is administered, without limitation. Examples of carriers are described herein and also in Remington: The Science and Practice of Pharmacy (Remington: The Science and Practice of Pharmacy, 23rd Edition, ISBN-13:978-0128200070).

The term “diluent” refers to chemical compounds that are used to dilute the compound of interest prior to delivery. Diluents can also serve to stabilize compounds. Non-limiting examples of diluents include starch, saccharides, disaccharides, sucrose, lactose, polysaccharides, cellulose, cellulose ethers, hydroxypropyl cellulose, sugar alcohols, xylitol, sorbitol, maltitol, microcrystalline cellulose, calcium or sodium carbonate, lactose, lactose monohydrate, dicalcium phosphate, cellulose, compressible sugars, dibasic calcium phosphate dehydrate, mannitol, and tribasic calcium phosphate.

The term “binder” when used herein relates to any pharmaceutically acceptable film which can be used to bind together the active and inert components of the carrier together to maintain cohesive and discrete portions. Non-limiting examples of binders include hydroxypropylcellulose, hydroxypropylmethylcellulose, povidone, copovidone, and ethyl cellulose.

The term “disintegrant” refers to a substance which, upon addition to a solid preparation, facilitates its break-up or disintegration after administration and permits the release of an active ingredient as efficiently as possible to allow for its rapid dissolution. Non-limiting examples of disintegrants include maize starch, sodium starch glycolate, croscarmellose sodium, modified corn starch, sodium carboxymethyl starch, crospovidone, pregelatinized starch, and alginic acid.

The term “lubricant” refers to an excipient which is added to a powder blend to prevent the compacted powder mass from sticking to the equipment during the tableting or encapsulation process. It aids the ejection of the tablet form the dies, and can improve powder flow. Non-limiting examples of lubricants include magnesium stearate, stearic acid, silica, fats, calcium stearate, polyethylene glycol, sodium stearyl fumarate, or talc; and solubilizers such as fatty acids including lauric acid, oleic acid, and C8/C10 fatty acid.

The term “film coating” refers to a thin, uniform, film on the surface of a substrate (e.g. tablet). Film coatings are particularly useful for protecting the active ingredient from photolytic degradation. Non-limiting examples of film coatings include polyvinylalcohol based, hydroxyethylcellulose, hydroxypropylmethylcellulose, sodium carboxymethylcellulose, polyethylene glycol 4000 and cellulose acetate phthalate film coatings.

The term “glidant” as used herein is intended to mean agents used in tablet and capsule formulations to improve flow-properties during tablet compression and to produce an anti-caking effect. Non-limiting examples of glidants include colloidal silicon dioxide, talc, fumed silica, starch, starch derivatives, and bentonite.

The term “effective amount” or “therapeutically effective amount” refers to an amount that is sufficient to effect treatment, as defined herein, when administered to a mammal in need of such treatment. The therapeutically effective amount will vary depending upon the patient being treated, the weight and age of the patient, the severity of the disease condition, the manner of administration and the like, which can readily be determined by one of ordinary skill in the art.

The term “unit dosage forms” or “pharmaceutical dosage forms” refers to physically discrete units suitable as unitary dosages for human patients and other mammals, each unit containing a predetermined quantity of active material calculated to produce the desired therapeutic effect, in association with a suitable pharmaceutical excipient (e.g., a tablet).

The term “treatment” or “treating,” to the extent it relates to a disease or condition includes preventing the disease or condition from occurring, inhibiting the disease or condition, eliminating the disease or condition, and/or relieving one or more symptoms of the disease or condition.

The term “% w/w” as used herein refers to the weight of a component based on the total weight of a composition comprising the component. For example, if component A is present in an amount of 50% w/w in a 100 mg composition, component A is present in an amount of 50 mg.

Unless specifically stated otherwise, where a compound may assume alternative tautomeric, regioisomeric and/or stereoisomeric forms, all alternative isomers, are intended to be encompassed within the scope of the claimed subject matter. For example, when a compound is described as a particular optical isomer D- or L-, it is intended that both optical isomers be encompassed herein. For example, where a compound is described as having one of two tautomeric forms, it is intended that both tautomers be encompassed herein. Thus, the compounds provided herein may be enantiomerically pure, or be stereoisomeric or diastereomeric mixtures. The compounds provided herein may contain chiral centers. Such chiral centers may be of either the (R) or (S) configurations, or may be a mixture thereof. The chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

The present disclosure also encompasses all suitable isotopic variants of the compounds according to the present disclosure, whether radioactive or not. An isotopic variant of a compound according to the present disclosure is understood to mean a compound in which at least one atom within the compound according to the present disclosure has been exchanged for another atom of the same atomic number, but with a different atomic mass than the atomic mass which usually or predominantly occurs in nature. Examples of isotopes which can be incorporated into a compound according to the present disclosure are those of hydrogen, carbon, nitrogen, oxygen, fluorine, chlorine, bromine and iodine, such as ²H (deuterium), ³H (tritium), ¹³C, ¹⁴C, ¹⁵N, ¹⁷O, ¹⁸O, ¹⁸F, ³⁶Cl, ⁸²Br, ¹²³I, ¹²⁴I, ¹²⁵I, ¹²⁹I and ¹³¹I. Particular isotopic variants of a compound according to the present disclosure, especially those in which one or more radioactive isotopes have been incorporated, may be beneficial, for example, for the examination of the mechanism of action or of the active compound distribution in the body. Compounds labelled with ³H, ¹⁴C and/or ¹⁸F isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required. In some embodiments, hydrogen atoms of the compounds described herein may be replaced with deuterium atoms. In certain embodiments, “deuterated” as applied to a chemical group and unless otherwise indicated, refers to a chemical group that is isotopically enriched with deuterium in an amount substantially greater than its natural abundance. Isotopic variants of the compounds according to the present disclosure can be prepared by various, including, for example, the methods described below and in the working examples, by using corresponding isotopic modifications of the particular reagents and/or starting compounds therein.

Thus, any of the embodiments described herein are meant to include a salt, a single stereoisomer, a mixture of stereoisomers and/or an isotopic form of the compounds.

Unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 10%, 9%, 8%, 7%, 6%, 5%, 4%, 3%, 2%, 1%, 0.5%, 0.4%, 0.3%, 0.25%, 0.2%, 0.1% or 0.05% of a given value or range. Unless otherwise specified, the term “about” means within plus or minus 10% of a the explicitly recited value, rounded either up or down to the nearest integer.

### 5. EXAMPLES

The Examples in this section are offered by way of illustration, and not by way of limitation. The examples can represent only some embodiments, and it should be understood that the following examples are illustrative and not limiting. All substituents, unless otherwise specified, are as previously defined. The reagents and starting materials are readily available to one of ordinary skill in the art. The specific synthetic steps for each of the routes described may be combined in different ways, or in conjunction with steps from different schemes, to prepare the compounds described herein.

### 5.1. Example 1: A 2×2 Factorial Mendelian Randomization Analysis of the Effect of Combined SGLT2 and CETP Genetic Variation on Diabetes Incidence in the UK Biobank

### Introduction

A post-hoc meta-analysis of data gathered from clinical trials of CETP inhibitors demonstrated that CETP inhibitor usage, either alone or combined with other lipid-lowering drugs, was associated with decreased incidence of diabetes (see, e.g., W. Masson et al., Therapy with cholesteryl ester transfer protein (CETP) inhibitors and diabetes risk, Diabetes & Metabolism, Volume 44, Issue 6, 2018, Pages 508-513). In the current study described below, it was investigated whether or not a combination of a CETP inhibitor and a SGLT2 inhibitor would lead to decreased incidence of diabetes when compared to SGLT2 inhibitor monotherapy through a 2×2 factorial Mendelian Randomization (MR) design using 233,765 individuals from the UK Biobank.

### Methods

Constructing CETP genetic score: To construct a genetic score to mimic the effects of CETP inhibitors, a score of 4 single nucleotide polymorphisms (SNPs) in the CETP region that are strongly correlated with HDL were built. The genetic score uses all available SNPs in the UK Biobank genotyping information that were included in the CETP score described in Ference et al. (Ference B A, Kastelein J J P, Ginsberg H N, et al. Association of Genetic Variants Related to CETP Inhibitors and Statins With Lipoprotein Levels and Cardiovascular Risk. JAMA. 2017; 318(10):947-956). A higher CETP genetic score mimics a greater degree of pharmaceutical CETP inhibition.

Constructing SGLT2 genetic score: To construct a genetic score to mimic the effects of SGLT2 inhibitors, a score of 2 single nucleotide polymorphisms (SNPs) in the SGLT2 region that are strongly correlated with SGLT2 expression were built. The genetic score uses all available SNPs in the UK Biobank genotyping information that were included in the SGLT2 score described in Katzmann et al. (Katzmann, J. L., Mason, A. M., März, W., Kleber, M. E., Niessner, A., Blüher, M., Speer, T. and Laufs, U. (2021), Genetic Variation in Sodium-glucose Cotransporter 2 and Heart Failure. Clin. Pharmacol. Ther., 110:149-158). A higher SGLT2 genetic score mimics a greater degree of pharmaceutical SGLT2 inhibition.

Included individuals: For the 2×2 factorial MR analysis, all individuals in the UK Biobank that have genotyping information containing all SNPs needed to construct the genetic scores were included, as well as all biomarker values. Furthermore, only individuals of British descent were included in the analysis to control for population stratification bias. In total, 233,765 individuals fulfilled all inclusion criteria.

Separation into groups: Individuals were separated into two groups based on whether their CETP genetic score was greater than or less than the median CETP score. In each group, individuals were then separated into two additional groups based on whether their SGLT2 genetic score was greater than or less than the median SGLT2 genetic score. In total, four groups were formed, and a breakdown of each is presented in Table 1 below.

TABLE 1
Groups for Study

| | |
|---|---|
| Group 1: Control | Group 2: SGLT2 Inhibitor Monotherapy |
| Low CETP & Low SGLT2 | Low CETP & High SGLT2 |
| n = 57114 | n = 45491 |
| Glycated Hemoglobin = 35.07464(3.577379) | Glycated Hemoglobin = 35.03245(3.528246) |
| HDL = 1.394616(0.356581) | HDL = 1.398347(0.3583918) |
| Age = 56.84405(8.016776) | Age = 56.90734(7.97928) |
| BMI = 27.35826(4.703198) | BMI = 27.42289(4.699067) |
| BP = 140.0006(19.5886) | BP = 140.3652(19.71686) |
| ApoB = 1.042628(0.2396645) | ApoB = 1.044162(0.23833) |
| Weight = 78.1888(15.75372) | Weight = 78.23467(15.73402) |
| TG = 1.726181(0.9076712) | TG = 1.729812(0.9107276) |
| LDL = 3.596809(0.8646178) | LDL = 3.604041(0.863603) |
| Diabetes = 0.06663865 | Diabetes = 0.06625486 |
| Male = 0.4607627 | Male = 0.4586841 |
| Group 3: CETP Inhibitor Monotherapy | Group 4: Combination Therapy |
| High CETP & Low SGLT2 | High CETP & High SGLT2 |
| n = 72986 | n = 58174 |
| Glycated Hemoglobin = 35.05359(3.542238) | Glycated Hemoglobin = 34.92626(2.379608) |
| HDL = 1.501572(0.3913476) | HDL = 1.504738(0.391744) |
| Age = 56.85901(7.993711) | Age = 56.8183(8.020908) |
| BMI = 27.36337(4.703893) | BMI = 27.33449(4.707718) |
| BP = 139.9734(19.61371) | BP = 140.0963(19.6231) |
| ApoB = 1.03136(0.2363253) | ApoB = 1.028937(0.2367295) |
| Weight = 78.16706(15.75968) | Weight = 78.02971(15.77483) |
| TG = 1.6939(0.8923357) | TG = 1.689387(0.8897538) |
| LDL = 3.572271(0.8543885) | LDL = 3.563981(0.8545262) |
| Diabetes = 0.06512208 | Diabetes = 0.06251934 |
| Male = 0.4603212 | Male = 0.4599477 |

As used herein, Group 1 will be referred to as the “control group,” Group 2 will be referred to as the “SGLT2 monotherapy group,” Group 3 will be referred to as the “CETP monotherapy group,” and Group 4 will be referred to as the “combination therapy group.” This nomenclature is adopted because these are the treatments that each group represents, but note that the changes in HDL and glycated hemoglobin mediated by separation into groups based on genetic score differ in magnitude than the actual pharmaceutical treatments they are intended to help investigate.

Statistical Analysis: Comparisons between quantitative traits in each group were made using one-way ANOVA. When the ANOVA detects differences between groups, each group was compared to every other group pairwise using one-way ANOVA to characterize the differences between the groups. Comparison of gender composition was performed using chi squared. Comparisons in incidence of diabetes between groups was performed using logistic regression.

### Results and Discussion

The ANOVA conducted between the four groups with respect to HDL yielded an overall p-value (p<0.00001), suggesting that the groups differ significantly in their mean HDL value. We characterized the specific differences using pairwise ANOVA between each of the groups. The results are given below in Table 2:

TABLE 2
ANOVA analysis between groups with respect to HDL

HDL: p < 0.00001:

| | |
|---|---|
| Groups 1 and 2: p = 0.096833717 | Groups 2 and 3: p = 0 |
| Groups 1 and 3: p = 0 | Groups 2 and 4: p = 0 |
| Groups 1 and 4: p = 0 | Groups 3 and 4: p = 0.145723031 |

If the CETP score functioned as intended, it would be expected to see significant differences between control and CETP monotherapy, which was observed (Groups 1&3; p=0). It was observed that HDL was significantly different between Groups 1&3 (control vs CETP inhibitor monotherapy), 1&4 (control vs combination therapy), 2&3 (SGLT2 inhibitor monotherapy vs CETP inhibitor monotherapy), and 2&4 (SGLT2 inhibitor monotherapy vs combination therapy). These results indicate that separation of HDL between groups is achieved in a pattern consistent with CETP inhibitor therapy, and that the SGLT2 genetic score functions as intended. These results also suggest that SGLT2 inhibitors have no impact on HDL levels. The same pattern of statistical significance and opposite effect direction was observed for ApoB, LDL, and TG, which are the other biomarkers CETP genetic scores have been known to correlate with (see, e.g., Tables 9-11 under “Other Regression Data” below).

The ANOVA conducted between the four groups with respect to glycated hemoglobin similarly yielded an overall p-value (p<0.00001), suggesting significant differences between groups. The results are given below in Table 3:

TABLE 3
ANOVA analysis between groups with
respect to glycated hemoglobin

Glycated hemoglobin: p < 0.00001:

| | |
|---|---|
| Groups 1 and 2: p = 0.0359155 | Groups 2 and 3: p = 0.4574384 |
| Groups 1 and 3: p = 0.02759712 | Groups 2 and 4: p = 0.002838989 |
| Groups 1 and 4: p = 5.486184e−07 | Groups 3 and 4: p = 0.0005814967 |

If the SGLT2 score functioned as intended, it would be expected to see significant differences between control and SGLT2 monotherapy, which was observed (Groups 1&2; p=0.035). However, significant differences in glycated hemoglobin levels were also observed between groups 1&3, 1&4, 2&4, and 3&4. The CETP genetic score is also correlated with glycated hemoglobin, with CETP inhibitor monotherapy leading to a significant reduction. Notably, there was a significant reduction in glycated hemoglobin levels between Groups 2&4 (SGLT2 inhibitor monotherapy and combination therapy). These results indicate that combination therapy of SGLT2 and CETP inhibitors may be useful in achieving lower glycated hemoglobin levels than SGLT2 inhibitors alone.

When comparing the incidence of diabetes between the four groups, it was found that group 4 (combination therapy) was the only group with a significant reduction in incidence of diabetes compared to control (OR=0.934; p=0.00222). It was also found that group 4 had a significantly lower incidence of diabetes than group 3 (OR=0.969; p=0.00745) and group 2 (OR=0.957; p=0.02785). The decrease in diabetes incidence in group 4 compared to all other groups suggests that genetically mediated CETP and SGLT2 inhibition confers greater protection against the development of diabetes than genetically mediated inhibition of each individually. These results are summarized in Tables 4-6 below:

TABLE 4
Regression against Group 1

| | Group 2 | Group 3 | Group 4 |
|---|---|---|---|
| LOR | −0.006187 | −0.024645 | −0.068213 |
| p-value | 0.40316 | 0.13676 | 0.00222 |

TABLE 5
Comparison of Group 2 and 4

| | Group 4 |
|---|---|
| LOR | −0.03101 |
| p-value | 0.00745 |

TABLE 6
Comparison of Group 3 and 4

| | Group 4 |
|---|---|
| LOR | −0.04357 |
| p-value | 0.02785 |

The combined observations of decreased glycated hemoglobin and decreased incidence of diabetes between the combination therapy group and all others suggest that targeting CETP and SGLT2 simultaneously with pharmaceutical interventions will achieve increased glycemic control compared to targeting CETP or SGLT2 individually.

### Other Regression Data

The p-values from other regressions performed are given below and in Tables 7-11:
Age: p=0.3485
Sex: p=0.922937
Weight: p=0.1622; not significant.

TABLE 7
ANOVA analysis between groups with respect to BMI

BMI: p = 0.0240

| | |
|---|---|
| Groups 1 and 2: p = 0.02868823 | Groups 2 and 3: p = 0.03404580 |
| Groups 1 and 3: p = 0.84580846 | Groups 2 and 4: p = 0.002671235 |
| Groups 1 and 4: p = 0.39112817 | Groups 3 and 4: p = 0.26953163 |

TABLE 8
ANOVA analysis between groups with respect to BP

BP: p = 0.0053

| | |
|---|---|
| Groups 1 and 2: p = 0.003170139 | Groups 2 and 3: p = 0.0008574715 |
| Groups 1 and 3: p = 0.80322876 | Groups 2 and 4: p = 0.02901528 |
| Groups 1 and 4: p = 0.40729672 | Groups 3 and 4: p = 0.2593790 |

TABLE 9
ANOVA analysis between groups with respect to ApoB

ApoB: p < 0.00001

| | |
|---|---|
| Groups 1 and 2: p = 0.306662195 | Groups 2 and 3: p = 1.892183e−19 |
| Groups 1 and 3: p = 2.549734e−17 | Groups 2 and 4: p = 1.386075e−24 |
| Groups 1 and 4: p = 1.759157e−22 | Groups 3 and 4: p = 0.065251383 |

TABLE 10
ANOVA analysis between groups with respect to TG

TG: p < 0.00001

| | |
|---|---|
| Groups 1 and 2: p = 0.590532109 | Groups 2 and 3: p = 1.465046e−10 |
| Groups 1 and 3: p = 3.928793e−10 | Groups 2 and 4: p = 2.006882e−11 |
| Groups 1 and 4: p = 5.390325e−11 | Groups 3 and 4: p = 0.509293479 |

TABLE 11
ANOVA analysis between groups with respect to LDL

LDL: p < 0.00001

| | |
|---|---|
| Groups 1 and 2: p = 0.182860661 | Groups 2 and 3: p = 6.286311e−10 |
| Groups 1 and 3: p = 3.288490e−07 | Groups 2 and 4: p = 9.713143e−14 |
| Groups 1 and 4: p = 9.032990e−11 | Groups 3 and 4: p = 0.080882956 |

### Sensitivity Analysis

As indicated herein above, the SGLT2 score functioned as intended with respect to glycated hemoglobin, correlating with reduced glycated hemoglobin just as SGLT2 inhibition correlates to decreased glycated hemoglobin. However, the SGLT2 score was correlated with an increase in BMI and blood pressure between Groups 1&2, which is the opposite of the effect SGLT2 inhibitors are observed to have in practice.

To investigate this phenomenon further, a sensitivity analysis was conducted using linear regression (for glycated hemoglobin) and logistic regression (for diabetes) and included BMI, blood pressure, weight, age, and sex as covariates to isolate the effect of genetic score independent of these variables. The difference between Groups 2&4 and 3&4 was reassessed in glycated hemoglobin and diabetes under this analytical method. The results are presented below in Tables 12 and 13:

TABLE 12
Sensitivity Analysis of Glycated Hemoglobin

| | |
|---|---|
| Groups 2&4 | p = 0.0393 |
| Groups 3&4 | p = 0.0016 |

TABLE 13
Sensitivity Analysis of Diabetes Incidence

| | Groups 2&4 | Groups 3&4 |
|---|---|---|
| OR | 0.946 | 0.958 |
| p-value | 0.01905 | 0.03525 |

In summary, the results of the sensitivity analysis shown in Tables 12 and 13 are consistent with the results from the analysis described herein above (e.g., in the Results and Discussion section). The significance of the tests with respect to glycated hemoglobin and diabetes incidence are not affected by BMI, BP, weight, age, or sex.

### 5.2. Example 2: Preparation of Fixed Dose Tablet

The following examples are illustrative procedures of how obicetrapib and SGLT2 fixed dose combination tablets described herein can be prepared and tested.

### 5.2.1. Example 2A: Composition

Exemplary pharmaceutical dosage forms (e.g., as disclosed herein) are formulated as a 6 mm diameter, white, film-coated, round, biconvex tablet with no identifying markings. Tablets can be manufactured in the dosage strengths shown in tables 14-17 below (dose of obicetrapib expressed as the calcium salt).

A complete description of the components and quantitative composition of fixed dose tablets including obicetrapib 5 mg, or 10 mg, and a SGLT2 inhibitor are provided in tables 14-17 (quantities expressed as % w/w)

TABLE 14
Fixed dose formulation of obicetrapib and empagliflozin

| | Quantity | Quantity | Quantity | Quantity | |
|---|---|---|---|---|---|
| Component | (% w/w) | (% w/w) | (% w/w) | (% w/w) | |
| Obicetrapib [1] | 4.42 | 3.91 | 8.85 | 7.81 | Active (a) |
| Empagliflozin [2] | 8.85 | 19.53 | 8.85 | 19.53 | Active (b) |
| Microcrystalline cellulose | 57.52 | 50.78 | 53.10 | 46.88 | diluent |
| Mannitol | 20.35 | 18.00 | 20.35 | 18.00 | diluent |
| Sodium starch glycolate | 4.42 | 3.91 | 4.42 | 3.91 | disintegrant |
| Colloidal silicon dioxide | 0.88 | 0.78 | 0.88 | 0.78 | glidant |
| Magnesium stearate | 0.88 | 0.78 | 0.88 | 0.78 | lubricant |
| Opadry ® II white 32K280000 | 2.65 | 2.34 | 2.65 | 2.34 | coating |
| containing [3]: | | | | | |
| Hypromellose (HPMC 2910) | | | | | |
| Lactose monohydrate | | | | | |
| Titanium dioxide | | | | | |
| Triacetin | | | | | |

[1]. The obicetrapib is expressed as the calcium salt. The actual quantity of obicetrapib used may be adjusted (microcrystalline cellulose loading adjusted accordingly) based on the purity of each batch of drug substance.
[2]. The actual quantity of empagliflozin used may be adjusted (microcrystalline cellulose loading adjusted accordingly) based on the purity of each batch of drug substance.
[3]. Target weight of film coating applied to cores, amount will vary depending on total core weight.

TABLE 15
Fixed dose formulation of obicetrapib and ertugliflozin

| | Quantity | Quantity | Quantity | Quantity | |
|---|---|---|---|---|---|
| Component | (% w/w) | (% w/w) | (% w/w) | (% w/w) | |
| Obicetrapib [1] | 4.63 | 4.24 | 9.26 | 8.45 | Active (a) |
| Ertugliflozin [2] | 4.63 | 12.71 | 4.63 | 12.71 | Active (b) |
| Microcrystalline cellulose | 60.19 | 55.08 | 55.56 | 50.85 | diluent |
| Mannitol | 21.30 | 19.49 | 21.30 | 19.49 | diluent |
| Sodium starch glycolate | 4.63 | 4.24 | 4.63 | 4.24 | disintegrant |
| Colloidal silicon dioxide | 0.93 | 0.85 | 0.93 | 0.85 | glidant |
| Magnesium stearate | 0.93 | 0.85 | 0.93 | 0.85 | lubricant |
| Opadry ® II white 32K280000 | 2.78 | 2.54 | 2.78 | 2.54 | coating |
| containing [3]: | | | | | |
| Hypromellose (HPMC 2910) | | | | | |
| Lactose monohydrate | | | | | |
| Titanium dioxide | | | | | |
| Triacetin | | | | | |

[1]. The obicetrapib is expressed as the calcium salt. The actual quantity of obicetrapib used may be adjusted (microcrystalline cellulose loading adjusted accordingly) based on the purity of each batch of drug substance.
[2]. The actual quantity of empagliflozin used may be adjusted (microcrystalline cellulose loading adjusted accordingly) based on the purity of each batch of drug substance.
[3]. Target weight of film coating applied to cores, amount will vary depending on total core weight.

TABLE 16
Fixed dose formulation of obicetrapib and dapagliflozin

| | Quantity | Quantity | Quantity | Quantity | |
|---|---|---|---|---|---|
| Component | (% w/w) | (% w/w) | (% w/w) | (% w/w) | |
| Obicetrapib [1] | 4.63 | 4.42 | 9.26 | 8.85 | Active (a) |
| dapagliflozin [2] | 4.63 | 8.85 | 4.63 | 8.85 | Active (b) |
| Microcrystalline cellulose | 60.19 | 57.52 | 55.56 | 53.10 | diluent |
| Mannitol | 21.30 | 20.35 | 21.30 | 20.35 | diluent |
| Sodium starch glycolate | 4.63 | 4.42 | 4.63 | 4.42 | disintegrant |
| Colloidal silicon dioxide | 0.93 | 0.88 | 0.93 | 0.88 | glidant |
| Magnesium stearate | 0.93 | 0.88 | 0.93 | 0.88 | lubricant |
| Opadry ® II white 32K280000 | 2.78 | 2.65 | 2.78 | 2.65 | coating |
| containing [3]: | | | | | |
| Hypromellose (HPMC 2910) | | | | | |
| Lactose monohydrate | | | | | |
| Titanium dioxide | | | | | |
| Triacetin | | | | | |

[1]. The obicetrapib is expressed as the calcium salt. The actual quantity of obicetrapib used may be adjusted (microcrystalline cellulose loading adjusted accordingly) based on the purity of each batch of drug substance.
[2]. The actual quantity of empagliflozin used may be adjusted (microcrystalline cellulose loading adjusted accordingly) based on the purity of each batch of drug substance.
[3]. Target weight of film coating applied to cores, amount will vary depending on total core weight.

TABLE 17
Fixed dose formulation of obicetrapib and canagliflozin

| | Quantity | Quantity | Quantity | Quantity | |
|---|---|---|---|---|---|
| Component | (% w/w) | (% w/w) | (% w/w) | (% w/w) | |
| Obicetrapib [1] | 2.46 | 1.24 | 4.92 | 2.48 | Active (a) |
| canagliflozin [2] | 49.26 | 74.44 | 49.26 | 74.44 | Active (b) |
| Microcrystalline cellulose | 32.02 | 16.13 | 29.56 | 14.89 | diluent |
| Mannitol | 11.33 | 5.71 | 11.33 | 5.71 | diluent |
| Sodium starch glycolate | 2.46 | 1.24 | 2.46 | 1.24 | disintegrant |
| Colloidal silicon dioxide | 0.49 | 0.25 | 0.49 | 0.25 | glidant |
| Magnesium stearate | 0.49 | 0.25 | 0.49 | 0.25 | lubricant |
| Opadry ® II white 32K280000 | 1.48 | 0.74 | 1.48 | 0.74 | coating |
| containing: | | | | | |
| Hypromellose (HPMC 2910) | | | | | |
| Lactose monohydrate | | | | | |
| Titanium dioxide | | | | | |
| Triacetin | | | | | |

[1]. The obicetrapib is expressed as the calcium salt. The actual quantity of obicetrapib used may be adjusted (microcrystalline cellulose loading adjusted accordingly) based on the purity of each batch of drug substance.
[2]. The actual quantity of empagliflozin used may be adjusted (microcrystalline cellulose loading adjusted accordingly) based on the purity of each batch of drug substance.
[3]. Target weight of film coating applied to cores, amount will vary depending on total core weight.

### 5.2.1. Example 2B: Preparation

A general manufacturing process for the fixed dose tablets described above by direct compression is described as follows:
(1) Obicetrapib and the SGLT2 inhibitor are blended with microcrystalline cellulose, mannitol, sodium starch glycolate, and colloidal silicon dioxide in a mixer to obtain a pre-mixture;
(2) magnesium stearate is added to the pre-mixture of step (1) and mixing continued;
(3) tableting the final mixture of step (1) by compressing it into 6 mm biconvex tablet cores on a suitable tablet press;
(4) Film-coating the 6 mm biconvex tablet cores with a proprietary aesthetic film coating.

### 6. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. A pharmaceutical composition comprising:
a) a therapeutically effective amount of obicetrapib, or a pharmaceutically acceptable salt thereof; and
b) a therapeutically effective amount of at least one SGLT2 inhibitor, or a pharmaceutically acceptable salt thereof.

2. The pharmaceutical composition according to claim 1, wherein the obicetrapib is a calcium salt.

3. The pharmaceutical composition according to claim 1 or 2, wherein the SGLT2 inhibitor is selected from canagliflozin, dapagliflozin, ertugliflozin, empagliflozin, bexagliflozin, tofogliflozin, ipragliflozin, luseogliflozin, remogliflozin etabonate, sergliflozin etabonate, and sotagliflozin.

4. The pharmaceutical composition according to any one of claims 1 to 3, wherein the SGLT2 inhibitor is selected from canagliflozin, dapagliflozin, ertugliflozin, and empagliflozin.

5. The pharmaceutical composition according to claim 3 or 4, wherein the SGLT2 inhibitor is empagliflozin.

6. The pharmaceutical composition according to claim 3 or 4, wherein the SGLT2 inhibitor is dapagliflozin.

7. The pharmaceutical composition according to claim 3 or 4, wherein the SGLT2 inhibitor is canagliflozin.

8. The pharmaceutical composition according to claim 3 or 4, wherein the SGLT2 inhibitor is ertugliflozin.

9. The pharmaceutical composition according to any one of claims 1 to 8, further comprising one or more additional active agents.

10. The pharmaceutical composition according to claim 9, wherein the one or more additional active agents is selected from metformin and a DDP-4 inhibitor, or pharmaceutically acceptable salts thereof.

11. The pharmaceutical composition according to claim 10, wherein the DDP-4 inhibitor is linagliptin.

12. The pharmaceutical composition according to any one of claims 1 to 11, comprising from 1% to 25% w/w of obicetrapib.

13. The pharmaceutical composition according to claim 12, comprising from 1% to 10% w/w of obicetrapib.

14. The pharmaceutical composition according to claim 13, comprising about 5% w/w of obicetrapib.

15. The pharmaceutical composition according to claim 13, comprising about 10% w/w of obicetrapib.

16. The pharmaceutical composition according to any one of claims 1 to 15, comprising from 5% to 50% w/w of the SGLT2 inhibitor.

17. The pharmaceutical composition according to claim 16, comprising from 5% to 25% w/w of the SGLT2 inhibitor.

18. The pharmaceutical composition according to any one of claims 1 to 17, further comprising one or more of a diluent, a disintegrant, a glidant, a filler, a lubricant, and any combination thereof.

19. The pharmaceutical composition according to claim 18, wherein the diluent is selected from the group consisting of dicalcium phosphate, cellulose, microcrystalline cellulose, compressible sugars, dibasic calcium phosphate dehydrate, lactose, lactose monohydrate, lactose anhydrous, mannitol, tribasic calcium phosphate, and combinations thereof.

20. The pharmaceutical composition according to claim 18 or 19, wherein the disintegrant is selected from the group consisting of croscarmellose sodium, crospovidone, modified corn starch, pregelatinized starch, sodium starch glycolate, and combinations thereof.

21. The pharmaceutical composition according to any one of claims 18 to 20, wherein the glidant is selected from the group consisting of colloidal silicon dioxide, talc, and combinations thereof.

22. The pharmaceutical composition according to any one of claims 18 to 21, wherein the lubricant is selected from the group consisting of calcium stearate, magnesium stearate, polyethylene glycol, sodium stearyl fumarate, stearic acid, and combinations thereof.

23. The pharmaceutical composition according to any one of claims 1 to 22, wherein the composition comprises:

| | Amount (% by |
|---|---|
| | weight) |
| Obicetrapib | 1-10 |
| SGTL2 inhibitor | 4-75 |
| one or more diluents | 5-85 |
| one or more disintegrants | 1-15 |
| one or more additional additives | up to100% |

24. The pharmaceutical composition according to claim 23, further comprising one or more lubricants.

25. The pharmaceutical composition according to claim 23 or 24, further comprising one or more glidants.

26. A pharmaceutical dosage form comprising the pharmaceutical composition of any one of claims 1 to 25.

27. The pharmaceutical dosage form of claim 26, wherein the obicetrapib is present in an amount of from 1 to 25 mg.

28. The pharmaceutical dosage form of claim 27, wherein the obicetrapib is present in an amount of from 1 to 10 mg.

29. The pharmaceutical dosage form according to claim 27, wherein the obicetrapib is present in an amount of 5 mg.

30. The pharmaceutical dosage form according to claim 27, wherein the obicetrapib is present in an amount of 10 mg.

31. The pharmaceutical dosage form according to any one of claims 26 to 30, wherein the SGLT2 inhibitor is present in an amount of from 1 to 300 mg.

32. The pharmaceutical dosage form according to claim 31, wherein the SGLT2 inhibitor is present in an amount of from 5 to 100 mg.

33. The pharmaceutical dosage form according to claim 31, wherein the SGLT2 inhibitor is present in an amount of from 5 to 50 mg.

34. The pharmaceutical dosage form according to claim 31, wherein the SGLT2 inhibitor is present in an amount of from 10 to 25 mg.

35. The pharmaceutical dosage form according to claim 34, wherein the SGLT2 inhibitor is empagliflozin.

36. The pharmaceutical dosage form according to claim 31, wherein the SGLT2 inhibitor is present in an amount of from 5 to 15 mg.

37. The pharmaceutical dosage form according to claim 36, wherein the SGLT2 inhibitor is ertugliflozin.

38. The pharmaceutical dosage form according to claim 31, wherein the SGLT2 inhibitor is present in an amount of from 5 to 10 mg.

39. The pharmaceutical dosage form according to claim 38, wherein the SGLT2 inhibitor is dapagliflozin.

40. The pharmaceutical dosage form according to claim 31, wherein the SGLT2 inhibitor is present in an amount of from 100 to 300 mg.

41. The pharmaceutical dosage form according to claim 40, wherein the SGLT2 inhibitor is canagliflozin.

42. The pharmaceutical dosage form according to claim 31, wherein the SGLT2 inhibitor is present in an amount of 5 mg.

43. The pharmaceutical dosage form according to claim 31, wherein the SGLT2 inhibitor is present in an amount of 10 mg.

44. The pharmaceutical dosage form according to claim 31, wherein the SGLT2 inhibitor is present in an amount of 15 mg.

45. The pharmaceutical dosage form according to claim 31, wherein the SGLT2 inhibitor is present in an amount of 25 mg.

46. The pharmaceutical dosage form according to claim 31, wherein the SGLT2 inhibitor is present in an amount of 100 mg.

47. The pharmaceutical dosage form according to claim 31, wherein the SGLT2 inhibitor is present in an amount of 300 mg.

48. The pharmaceutical dosage form according to claim 26, wherein the obicetrapib is present in an amount of from 5 to 10 mg; and the SGLT2 inhibitor is empagliflozin, present in an amount of from 10 to 25 mg.

49. The pharmaceutical dosage form according to claim 26, wherein the obicetrapib is present in an amount of from 5 to 10 mg; and the SGLT2 inhibitor is ertugliflozin, present in an amount of from 5 to 15 mg.

50. The pharmaceutical dosage form according to claim 26, wherein the obicetrapib is present in an amount of from 5 to 10 mg; and the SGLT2 inhibitor is dapagliflozin, present in an amount of from 5 to 10 mg.

51. The pharmaceutical dosage form according to claim 26, wherein the obicetrapib is present in an amount of from 5 to 10 mg; and the SGLT2 inhibitor is canagliflozin, present in an amount of from 100 to 300 mg.

52. The pharmaceutical dosage form according to any one of claims 26 to 51, wherein the dosage form is a solid dosage form.

53. The pharmaceutical dosage form according to claim 52, wherein the solid dosage form is a tablet.

54. The pharmaceutical dosage form according to claim 53, further comprising a film coating.

55. A method of treating or preventing a metabolic disorder, comprising administering to a subject having or at risk of developing a metabolic disorder a therapeutically effective amount of a pharmaceutical composition according to any one of claims 1 to 25, or a pharmaceutical dosage form of any one of claims 26 to 54.

56. The method of claim 55, wherein the metabolic disorder is selected from type 1 diabetes mellitus, type 2 diabetes mellitus, impaired glucose tolerance (IGT), impaired fasting blood glucose (IFG), hyperglycemia, postprandial hyperglycemia, obesity, and metabolic syndrome.

57. A method of treating or preventing a metabolic disorder in a subject who has or is at risk of developing a metabolic disorder, comprising:
administering a therapeutically effective amount of obicetrapib, or a pharmaceutically acceptable salt thereof; and
a therapeutically effective amount of at least one SGLT2 inhibitor, or a pharmaceutically acceptable salt thereof.

58. The method of claim 57, wherein the metabolic disorder is selected from type 1 diabetes mellitus, type 2 diabetes mellitus, impaired glucose tolerance (IGT), impaired fasting blood glucose (IFG), hyperglycemia, postprandial hyperglycemia, obesity, and metabolic syndrome.

59. The method according to claim 57 or 58, wherein the obicetrapib is a calcium salt.

60. The method of any one of claims 57 to 59, wherein the SGLT2 inhibitor is selected from canagliflozin, dapagliflozin, ertugliflozin, empagliflozin, bexagliflozin, tofogliflozin, ipragliflozin, luseogliflozin, remogliflozin etabonate, sergliflozin etabonate, and sotagliflozin.

61. The method of any one of claims 57 to 60, wherein the SGLT2 inhibitor is selected from canagliflozin, dapagliflozin, ertugliflozin, and empagliflozin.

62. The method of claim 60 or 61, wherein the SGLT2 inhibitor is empagliflozin.

63. The method of claim 60 or 61, wherein the SGLT2 inhibitor is dapagliflozin.

64. The method of claim 60 or 61, wherein the SGLT2 inhibitor is canagliflozin.

65. The method of claim 60 or 61, wherein the SGLT2 inhibitor is ertugliflozin.

66. The method of any one of claims 57 to 65, wherein the therapeutically effective amount of obicetrapib is from 1 to 25 mg per day.

67. The method of claim 66, wherein the therapeutically effective amount of obicetrapib is from 1 to 10 mg per day.

68. The method of claim 66, wherein the therapeutically effective amount of obicetrapib is 5 mg per day.

69. The method of claim 66, wherein the therapeutically effective amount of obicetrapib is 10 mg per day.

70. The method of any one of claims 57 to 61, wherein the therapeutically effective amount of the SGLT2 inhibitor is from 1 to 300 mg per day.

71. The method of claim 70, wherein the therapeutically effective amount of the SGLT2 inhibitor is from 5 to 100 mg per day.

72. The method of claim 70, wherein the therapeutically effective amount of the SGLT2 inhibitor is from 5 to 50 mg per day.

73. The method of claim 70, wherein the therapeutically effective amount of the SGLT2 inhibitor is from 10 to 25 mg per day.

74. The method of claim 70, wherein the therapeutically effective amount of the SGLT2 inhibitor is from 5 to 15 mg per day.

75. The method of claim 70, wherein the therapeutically effective amount of the SGLT2 inhibitor is from 5 to 10 mg per day.

76. The method of claim 70, wherein the therapeutically effective amount of the SGLT2 inhibitor is from 100 to 300 mg per day.

77. The method of claim 70, wherein the therapeutically effective amount of the SGLT2 inhibitor is 5 mg per day.

78. The method of claim 70, wherein the therapeutically effective amount of the SGLT2 inhibitor is 10 mg per day.

79. The method of claim 70, wherein the therapeutically effective amount of the SGLT2 inhibitor is 15 mg per day.

80. The method of claim 70, wherein the therapeutically effective amount of the SGLT2 inhibitor is 25 mg per day.

81. The method of claim 70, wherein the therapeutically effective amount of the SGLT2 inhibitor is 100 mg per day.

82. The method of claim 70, wherein the therapeutically effective amount of the SGLT2 inhibitor is 300 mg per day.

83. The method of claim 57, wherein the therapeutically effective amount of obicetrapib is from 5 to 10 mg per day; the therapeutically effective amount of the SGLT2 inhibitor is from 10 to 25 mg per day; and the SGLT2 inhibitor is empagliflozin.

84. The method of claim 57, wherein the therapeutically effective amount of obicetrapib is from 5 to 10 mg per day; the therapeutically effective amount of the SGLT2 inhibitor is from 5 to 15 mg per day; and the SGLT2 inhibitor is ertugliflozin.

85. The method of claim 57, wherein the therapeutically effective amount of obicetrapib is from 5 to 10 mg per day; the therapeutically effective amount of the SGLT2 inhibitor is from 5 to 10 mg per day; and the SGLT2 inhibitor is dapagliflozin.

86. The method of claim 57, wherein the therapeutically effective amount of obicetrapib is from 5 to 10 mg per day; the therapeutically effective amount of the SGLT2 inhibitor is from 100 to 300 mg per day; and the SGLT2 inhibitor is canagliflozin.
